(19) United States
(12) Patent Application Publication
(10) Pub. No.: US 2022/0395237 A1
(43) Pub. Date: Dec. 15, 2022

(54) AUTOMATIC AMBULATORY SUBJECT MONITORING

(71) Applicant: Medtonic, Inc., Minneapolis, MN (US)

(72) Inventors: Paul A. Iaizzo, White Bear Lake, MN (US); Timothy G. Laske, Shoreview, MN (US); Paul G. Krause, Mahtomedi, MN (US); Kevin T O'Brien, Minneapolis, MN (US); Alex E. Kelner, Mounds View, MN (US)

(21) Appl. No.: 17/806,373

(22) Filed: Jun. 10, 2022

Related U.S. Application Data
(60) Provisional application No. 63/209,895, filed on Jun. 11, 2021.

(51) Int. Cl. A61B 5/00

(52) U.S. Cl. CPC A61B 5/7282; A61B 5/0004; A61B 5/746; A61B 5/0022; A61B 5/743; A61B 5/7465; A61B 5/68; A61B 2503/40

(57) ABSTRACT

A system coupled to a non-human subject comprising communication circuitry configured to communicatively couple to a medical device coupled to the subject and a remote computing system, the medical device configured to monitor physiological data of the non-human subject and the remote computing system configured to store historical data for the non-human subject; processing circuitry; and a memory comprising instructions that, when executed by the processing circuitry, cause the processing circuitry to: receive the monitored physiological data from the medical device; determine whether the medical device indicates that the monitored physiological data is in satisfaction of at least one condition triggering an upload of at least a portion of the monitored physiological data; and in response to the determination, generate, for communication to the remote computing system, output data comprising the at least a portion of the monitored physiological data.

This application claims the benefit of U.S. Provisional Application Ser. No. 63/209,895, filed Jun. 11, 2021, the entire content of which is incorporated herein by reference.

### FIELD

This disclosure generally relates to systems including devices configured to monitor physiological signals and, more particularly, to monitoring of ambulatory subjects using such systems.

### BACKGROUND

A variety of devices are configured to configured to monitor physiological signals of a subject. Such devices include implantable or wearable medical devices, as well as a variety of wearable health or fitness tracking devices. The physiological signals sensed by such devices include as examples, electrocardiogram (ECG) signals, electroencephalogram (EEG) signals, respiration signals, impedance (e.g., perfusion) signals, activity and/or posture signals, heart or respiratory sound signals, pressure signals, blood oxygen saturation signals, body composition, and blood glucose or other blood constituent signals. In general, using these signals, such devices facilitate monitoring and evaluating health over a number of months or years, outside of a clinic setting.

In some cases, such devices are configured to detect (e.g., acute) health events based on the physiological signals, such as episodes of cardiac arrhythmia, myocardial infarction, stroke, or seizure. Example arrhythmia types include cardiac pause or arrest (e.g., asystole), bradycardia, atrial fibrillation (AF), atrial tachycardia (AT), ventricular tachycardia (VT), and ventricular fibrillation (VF). The devices may store ECG and other physiological signal data collected during a time period including an episode as episode data.

### SUMMARY

The research and development of the above-discussed physiological monitoring devices has been focused on human subjects, including activity engaged in by human subjects and events experienced by human subjects. However, the same devices can be used for monitoring non-humans, including wild and domesticated animals, such as those in research facilities, preclinical facilities, veterinary clinics, and/or sanctuaries or zoos.

Many aspects of animal physiology and behavior and human physiology and behavior may not be comparable; therefore, aspects of systems, techniques, and devices for monitoring and detecting human health events may not be applicable to all types of animals in various environmental settings. Moreover, human subjects can provide valuable evidence (e.g., reports of symptoms) to consider when evaluating the physiological data (e.g., for additional clues) while animal subjects in the wild mostly avoid human contact and/or cannot communicate. The systems, techniques, and devices described herein may implement rules that have been calibrated for animal physiology, and enable, for a medical device, additional health event monitoring capabilities including those that are adaptable for animals of any taxa.

In general, the disclosure describes systems, techniques, and devices for monitoring non-human (e.g., animal) subjects. Given the potential to gain important intelligence regarding physiology of the animal subjects, the systems, techniques, and devices described herein leverage wireless communications technologies (e.g., a transponder) in health event monitoring and detection. While a human subject's physiological data generally provides a number of clues regarding the subject's health (both in general and specific to certain class(es) of medical problems), non-human subjects may make it difficult to collect such data. Animals living in the wild often are far away from any human bystander (e.g., biologist) and/or modern communications infrastructure, impeding human efforts in tracking these animals and/or monitoring their physiological data for health events. The systems, techniques, and devices described herein capture the non-human subject's data, for example, using new or different rules, to enhance a medical device's event monitoring capabilities, thereby improving resource consumption of any electro-mechanical tracking equipment (e.g., radio telemetry hardware) used in transmitting the captured data on behalf of a remote computing system.

As described herein, the tracking equipment is part of a system operative to perform one or more techniques for enabling remote monitoring of non-human subjects. The radio telemetry hardware of this equipment includes a radio transmitter/receiver and an antenna contained in a housing and attached to the subject in some manner (e.g., via an animal collar). Conventionally, the radio telemetry hardware may be combined with a pulse collar that is placed around the subject's neck or leg, but any material capable of being attached to the subject should suffice for the systems and techniques described herein. In such instances, the pulse collar is configured to transmit (e.g., broadcast) various information including location data for the subject, allowing a human to find/follow the subject. Each data transmission of the pulse collar utilizes one or more resource capacities (e.g., stored electrical power). Although the transmitted subject data may prove useful, a given data transmission may prove unnecessary and/or redundant. Other operations performed by the pulse collar in conventional systems may consume additional resource capacities, thereby reducing available resource capacities for performing future data transmissions. This includes the pulse collar regularly waking itself up (e.g., every five minutes) to detect communications equipment within a range for a successful data transmission.

There are a number of additional causes behind a conventional pulse collar's inefficient execution of its data transmission (e.g., upload) functionality and, in the wild, its short operating life. For at least these reasons, the conventional pulse collar is prone to non-functioning or malfunctioning due to running out of power and/or other resources. The present disclosure provides one or more techniques for improving resource utilization efficiency by a pulse collar such that the pulse collar, implementing techniques of this disclosure, consumes fewer resource capacities to successfully transmit data and if unnecessary, foregoes the given data transmission altogether.

In one example, a computing device communicatively coupled to a medical device of a non-human subject includes processing circuitry; and a memory includes receive monitored physiological data from the medical device configured to monitor the physiological data of the non-human subject; determine whether the medical device indicates that the monitored physiological data is in satisfaction of at least one condition triggering an upload of at least a portion of the monitored physiological data to a remote computing system configured to store historical data for the non-human subject; and in response to the determination, generate, for communication to the remote computing system, output data comprising the at least a portion of the monitored physiological data.

In another example, a medical device for a non-human subject includes one or more sensors configured to sense one or more physiological signals of the non-human subject; sensing circuitry configured to generate physiological data based on the one or more sensed physiological signals; communication circuitry configured to be communicatively coupled to a computing device, wherein the computing device is coupled to the non-human subject or a user; processing circuitry; and a memory includes determine that the physiological data is in satisfaction of at least one condition triggering an upload of the physiological data, wherein the at least one condition comprises a threshold defining a minimum acceleration; in response to the determination that accelerometer data satisfies at least one threshold, generate, for communication to the computing device, output data comprising the physiological data.

In another example, a method performed by processing circuitry of a computing device communicatively coupled to a medical device for a non-human subject includes receiving physiological data from the medical device, the medical device configured to monitor the physiological data of the non-human subject; determining whether the monitored physiological data is in satisfaction of at least one condition triggering an upload of at least a portion of the monitored physiological data to a remote computing system configured to store historical data for the non-human subject; and in response to determining that the monitored physiological data is in satisfaction of at least one condition, generating, for communication to the remote computing system via communication circuitry, output data comprising the at least a portion of the monitored physiological data.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a block diagram illustrating an example system configured to monitor a subject for health events in accordance with one or more techniques of this disclosure.

FIG. 2 is a block diagram illustrating an example configuration of a medical device that operates in accordance with one or more techniques of the present disclosure.

FIG. 3 is block diagram illustrating an example configuration of a computing device that operates in accordance with one or more techniques of the present disclosure.

FIG. 4 is a block diagram illustrating an example configuration of a health monitoring system that operates in accordance with one or more techniques of the present disclosure.

FIG. 5 is a flow diagram illustrating an example operation by a health monitoring system that operates in accordance with one or more techniques of the present disclosure.

FIG. 6 is a flow diagram illustrating an example interactive operation between a computing device and a computing system that operates in accordance with one or more techniques of the present disclosure.

Like reference characters refer to like elements throughout the figures and description.

### DETAILED DESCRIPTION

A variety of types of implantable and external devices are configured detect cardiac episodes (e.g., arrhythmias) and/or other health events based on sensed ECGs or other electrical signals and, in some cases, other physiological signals. External devices that may be used to non-invasively sense and monitor ECGs, EEGs, EMGs, and other physiological signals include wearable devices with electrodes configured to contact the skin of a non-human subject, such as patches or neck collars. The external devices may facilitate relatively longer-term monitoring of subject health during normal daily activities. The external devices may provide enhanced health event monitoring by leveraging network-accessible resources for various purposes including improved performance of native device functionality. For example, the external device may utilize remote computing system memory space and/or processing power to store, modify signal collection parameters, and/or analyze physiological data for the non-human subject (e.g., an animal).

Implantable medical devices (IMDs) also sense and monitor ECGs and other physiological signals, and detect health events such as episodes of arrhythmia, cardiac arrest, myocardial infarction, stroke, and seizure. Example IMDs include pacemakers and implantable cardioverter-defibrillators, which may be coupled to intravascular or extravascular leads, as well as pacemakers with housings configured for implantation within the heart, which may be leadless. Some IMDs do not provide therapy, such as implantable monitors. One example of such an IMD is the Reveal LINQ™ or LINQ II™ Insertable Cardiac Monitor (ICM), available from Medtronic plc, which may be inserted subcutaneously. Such IMDs may facilitate relatively longer-term monitoring of subjects during normal daily activities, and may periodically transmit collected data, e.g., episode data for detected arrhythmia episodes, to a remote monitoring system, such as the Medtronic Carelink™ Network. In some examples, glucose sensor systems may be implanted into the subject for monitoring other physiological signals.

Typical examples of the implantable and external devices are designed for human-computer interaction (HCI). The subject may be a non-human animal that cannot use device(s) configured for HCI. The present disclosure describes devices, systems, and techniques that are configured to mitigate (or overcome) shortcomings attributed to these devices when paired with non-human subjects. These devices, systems, and techniques realize, for the benefit of that non-human subject, at least some utility from the implantable and external devices despite those shortcomings. Often, interactivity with the non-human subject requires making judicious use of the limited physical space afforded to any computing devices to be attached to the non-human subject. The present disclosure describes, in addition to a collar-mounted transponder device, a harness for affixing the transponder device to the animal's body. A backpack or an alternate form of housing may be employed to contain the animal transponder device.

The devices, systems, and techniques described herein further enable interactivity between the external device and the IMD for the benefit of the non-human subject. The IMD may be implanted in a location that provides quality ECGs and/or other sensor signals and ensures reliable wireless connectivity to the external device. The present disclosure introduces, as an example external device for the non-human subject, an animal transponder device such as a collar-mounted receiver or another wearable device. An animal monitoring service running on another external device (e.g., a remote computing system in a cage or sanctuary setting, at a bating station, etc.) maintains historical datasets to the further benefit of the non-human subject.

The present disclosure describes a number of wireless communication technologies that the systems, techniques, and devices described herein may avail to obtain physiological data from non-human subjects and protect these subjects while in captivity or from certain dangers affecting the wild animal kingdom (e.g., poaching). The systems, techniques, and devices described herein may make advantageous use of wireless communication technologies, sensors, and networking (e.g., sensor networks) to obtain and then, process the non-human subject physiological data (e.g., electrocardiograms (EKGs), electroencephalograms (EGGs), electromyograms (EMGs), temperatures, impedances, position, activities, respiration, glucose levels, focal blood flow, tissue oxygenation, and/or the like). The above-mentioned animal collar-mounted transponder device (e.g., a pulse collar) may be configured to search for such communications equipment when a data transmission is requested by the remote computing system. Thus, control over the collar may be on-demand such that important physiological data can be delivered, in real-time, to any human in charge of the animal, for instance, if the wild animal is experiencing or has experienced a health event. Instead of waking up multiple times a day, the collar-mounted transponder device remains in a power-off mode or a low-power mode throughout the day(s), except for when a portion of the animal's data is requested by the remote computing system.

The present disclosure describes how the systems, techniques, and devices also benefit those monitoring the well-being of the subject. In some examples where the subject is a wild animal that receives monitoring via a collar-mounted transponder device and/or a medical device, a human in charge of caring for the wild animal (e.g., in a zoo or animal sanctuary) may, via vocal responses, answer queries corresponding to an imminent or occurring health event including an attack from a poacher or an unlicensed game hunter. As another benefit, the one or more computing devices may implement a rules engine (e.g., a model such as a mathematical model or a machine learning model) and incorporate the human caregiver's answers in a rules-based evaluation of the subject's physiological data; this is performed instead of using only the subject's physiological data in the rules-based evaluation.

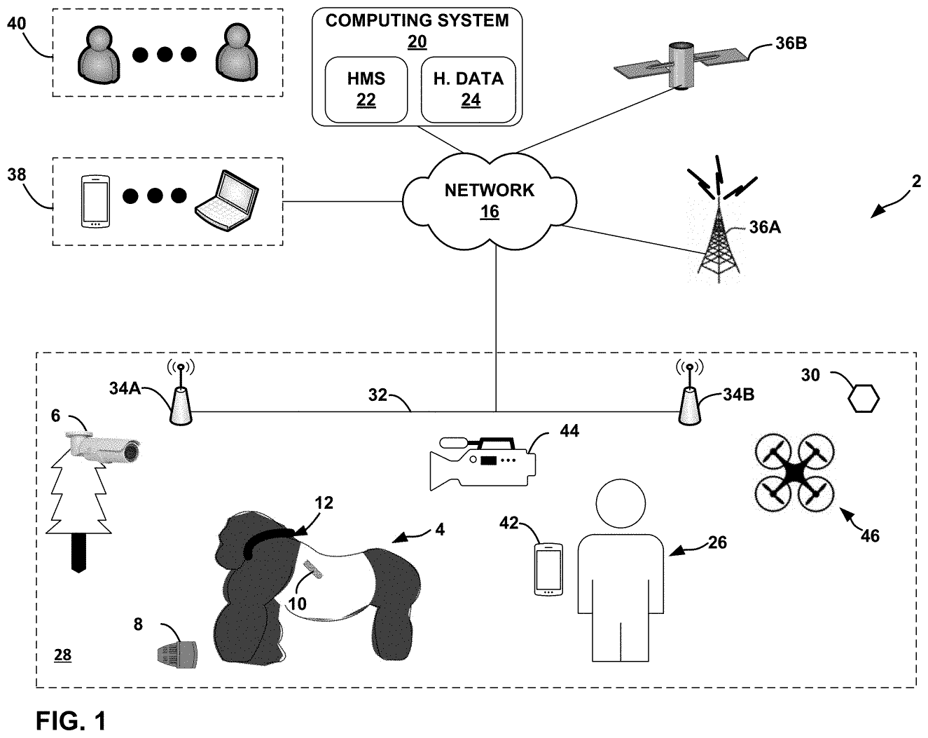

FIG. 1 is a block diagram illustrating an example system 2 configured to monitor a subject 4 for health events in accordance with one or more techniques of this disclosure. Devices of example system 2 generate various data during said monitoring, and any data generated for subject 4 is subject to being uploaded to a remote computing system for storage in historical data for that subject 4. In particular, example system 2 is configured to coordinate sensing of various physiological signals (e.g., physiological parameters) for subject 4 and then, direct transmission of desired physiological datasets to the remoted computing system.

As illustrated in FIG. 1, subject 4 may be a non-human subject, such as a wild animal, in a natural habitat away from human habitations and, accordingly, a number of environmental sensors may be employed to sense physiological parameters (e.g., measurements) and other physiological data. In addition to environmental sensors such as top-view camera 6 or ground sensor 8, medical device 10 and/or computing device 12 may be configured to monitor and record various data for environment 28. This may include sensing physiological data of subject 4 and, possibly, other non-human subjects in environment 28 (e.g., animals of a same species and/or animals of different species). Any form of a capture device, such as an image and/or video capture device, may be employed for camera 6. In some examples, a remote computing system, such as computing system 20, may instrument camera 6 to measure one or more characteristics of light data (e.g., infrared, ultraviolet, x-ray, and/or the like) in environment 28. In some examples, ground sensor 8 may a telemetry station, a proximity sensor, an infrared sensor, or another stationary sensor configured to record a specific data type. Drone 46 also may be configured with one or more sensors and to operate as a mobile sensory system. While the above environment sensors may be connected wirelessly to the Internet (via network 16), any one or more of these environment sensors may be network-accessible through a different networking technology as described herein. For instance, camera 6 or drone 46 may connected to network 16 via a cellular network configured with a known radio access technology (RAT).

In general, medical device 10 may employ various sensors to monitor one or more biological components such as a heart of subject 4, generate data associated with a physiology (e.g., cardiac physiology) of the one or more monitored biological components, and analyze the generated data for health events. In some examples, medical device 10 may be a type of a monitor (e.g., cardiac monitor, glucose monitor, brain monitor, and/or the like) configured with sensors (e.g., electrodes, glucose sensor, and/or the like) for capturing (e.g., cardiac) physiological signals (e.g., from the heart) of subject 4 and further configured with logic (e.g., a rules engine, a machine learning model, and/or the like) operative to detect various health events (e.g., cardiac episodes) affecting the (e.g., cardiac) physiology of subject 4. The logic may be programmed with software code and other data (e.g., configuration data) directed to performing the health event detection. Logic circuitry may implement the logic by coupling (e.g., encoding) the software code into hardware components. It should be noted that the logic may be updated (e.g., reprogrammed) with new software code and configuration data. It should be further noted that, in addition to medical device, other devices (e.g., computing device 12, computing system 20, IoT device(s) 30, computing device(s) 38, user device 42, drone 46, and/or the like) may be configured with same or similar logic and also identify health events in the (e.g., cardiac) physiological data.

As used herein, the terms “detect,” “detection,” and the like may refer to detection of a health event presently (at the time the data is collected) being experienced by subject 4, as well as detection based on the data that the condition of subject 4 is such that they have a suprathreshold likelihood of experiencing the event within a particular timeframe, e.g., prediction of the health event. The example techniques may be used with one or more subject sensing devices, e.g., IMD 10, which may be in wireless communication with one or more subject computing devices, e.g., subject computing device 12. Although not illustrated in FIG. 1, IMD 10 include electrodes and other sensors to sense physiological signals of subject 4 and may collect and store sensed physiological data based on the signals and detect episodes based on the data.

IMD 10 may be implanted outside of a thoracic cavity of subject 4 (e.g., subcutaneously in the pectoral location illustrated in FIG. 1). IMD 10 may be positioned near the sternum near or just below the level of the heart of subject 4, e.g., at least partially within the cardiac silhouette. In some examples, IMD 10 takes the form of the LINQ™ ICM. Although described primarily in the context of examples in which IMD 10 takes the form of an ICM, the techniques of this disclosure may be implemented in systems including any one or more implantable or external medical devices, including monitors, pacemakers, defibrillators, wearable external defibrillators, neurostimulators, or drug pumps. Furthermore, although described primarily in the context of examples including a single implanted subject sensing device, in some examples a system includes one or more subject sensing devices, which may be implanted within subject 4 or external to (e.g., worn by) subject 4.

Typical examples of IMD 10 and computing device 12 are designed for human-computer interaction (HCI). The subject may be a non-human animal and therefore, unable to use computing device(s) configured for HCI. To mitigate (or overcome) the non-human subject 4's handicap, the devices, systems, and techniques described herein enable interactivity between computing device 12 and IMD 10. As described herein, IMD 10 may be implanted in a location that provides accurate physiological data (e.g., ECGs and various physiological parameters) and ensures reliable wireless (e.g., BLUETOOTH) connectivity to computing device 12. As an example, computing device 12 for the non-human subject 4 may be an animal transponder device such as a collar-mounted receiver or another wearable device. An animal monitoring service, as an example of health monitoring service (HMS) 22 running on computing system 20, maintains historical data 24 to the further benefit of non-human subject 4. For example, the animal monitoring service may provide that historical data 24 for use in general or specific physiological research and/or in support of conservation/management and anti-poaching functionality.

IMD 10 and/or computing device 12 may include hardware/software components to implement device functionality and in some examples, to implement logic configured with a number of pre-selected conditions triggering an automatic data transmission (i.e., upload). IMB 10 and/or computing device 12 may be programed to transmit (e.g., upload) data on a regular schedule and/or based on available memory space. IMD 10 and/or computing device 12 may implement an alert system for human bystanders/caregivers or as an anti-poaching feature such that both IMB 10 and computing device 12 can detect the pre-selected conditions for triggering the automatic transmission of an alert. This transmission can include related information (e.g., physiological parameters) for confirming that a health event is cause for an alarm.

For IMD 10, a hardware/software component (e.g., logic) may be programed with various data for implementing one or more example conditions that, if satisfied, trigger the above-mentioned automatic transmission (e.g., upload) of information related to subject 4 including its physiological data. Each example condition represents a particular set of circumstances for which the upload of subject 4's physiological data somehow benefits subject 4 (and/or an organization/individual tracking subject 4). For one example condition, the above hardware/software component may prescribe one or more criterion for a sample of physiological data to satisfy in order to for that sample to qualify as (e.g., sufficient) evidence of a (e.g., potential) health event. The sample may cover a pre-determined time period (e.g., one or more time steps) of recorded physiological parameter signals. Each criterion of the above example condition may specify an evaluation methodology for verifying the sample as indicative of a valid health event.

To illustrate by way of an example implementation, the above hardware/software component may define (e.g., in a program) the one or more criterion in terms of one or more of the physiological parameters and, possibly, other parameters; for one example criterion, specific values of these parameters may be programmed into the above hardware/software component. Some example parameters values operate as (minimum or maximum) thresholds configured to distinguish actual occurrences of health events from non-events. In this example, some examples of the above-mentioned criterion are as follows: High or low heart rate, high or low impedance, high or low temperature or rapid change, rapid/extreme acceleration or lack of movement, rapid posture change or unusual posture, heart sounds, respiration rates, oxygen saturation, and/or the like. Setting one or more of these examples allows IMD 10 to quickly detect health events once they occur in the subject and furthermore, provides IMD 10 with ample time to contact emergency personnel. Given that the detected health events may be harmful to subject 4, the above hardware/software component extends an opportunity to an alert caregiver 26 for the subject 4. For instance, by setting a particular respiration rate (which may be detectable from QRS amplitude modulation) as a threshold for classifying a sample as a health event, the above hardware/software component may cause IMD 10 to output an alarm, thereby bringing attention to the health event occurring in subject 4.

The above hardware/software component of IMD 10 may be reprogrammed with new or modified criterion. Caregiver 26, via user device 42, and/or HMS 22, via computing system 20, may provide one or more parameter values to update (e.g., improve) a current configuration of IMD 10. As described herein, the new or modified criterion may be referred to as rules (e.g., for a rules engine) according to at least one embodiment. Over time, IMD 10 may be reprogrammed until at least a minimum level of performance (e.g., accuracy) is achieved. In some examples of the above hardware/software component of IMD 10, one or more physiology parameters of subject 4 may be evaluated for sufficient evidence of a valid health event. The hardware/software component of IMD 10 may determine that a valid health event most likely occurred in subject 4, for example, by determining at least one of a heart rate measurement exceeds a first threshold, an impedance measurement exceeds a second threshold, a temperature measurement exceeds a third threshold, a change in temperature per unit of time exceeds a fourth threshold, accelerometer data satisfies a fifth threshold, a respiration rate exceeds a sixth threshold, or an oxygen saturation satisfies a seventh threshold. It should be noted that additional criteria may be implemented by the hardware/software component of IMD 10 to determine whether a condition triggering the upload has been satisfied. The additional criteria may involve other physiological parameters. As an example, the hardware/software component of IMD 10 may compare pre-determined thresholds to a glucose level measurement or a blood pressure reading to determine whether a health event occurred.

For computing device 12, a hardware/software component (e.g., logic) may be programed with data for implementing one or more example conditions triggering an automatic transmission to a remote computing system (e.g., to complete the above-mentioned upload). Similar to IMD 10, each of the one or more example conditions represents a particular set of circumstances for which the upload of subject 4's physiological data somehow benefits subject 4 (and/or an organization/individual tracking subject 4) and each example condition establishes one or more criterion for a sample of physiological data to qualify as an incident of a potential health event. Therefore, IMD 10 may define an example condition using values of various physiological parameters such that the parameter values operate as (minimum or maximum) thresholds to distinguish actual occurrences of health events from non-events.

Examples of certain conditions that the hardware/software component may implement are as follows: Sudden acceleration; excess velocity; a lack of movement for a prescribed amount of time; GPS location or altitude out of prescribed range; loss of communication with implantable device; certain sounds; and image recognition. For instance, the hardware/software component of IMD may define an occurrence of sudden acceleration using movement data from one or more accelerometers and then, proceed to classify any sample exceeding that movement data as potential health events. Other sensors may record acoustic data that may be leveraged for health event detection in subject 4. As another example, the hardware/software component of IMD 10 may identify a specific heart sound indicating a cardiac episode in subject 4.

Among the sensors employed by computing device 12, a camera may be directed to record image data for IMD 10. The camera may be a local (e.g., internal) camera unit for computing device 12 and/or IMD 10 may capture images (e.g., images depicting subject 4) for an analysis, for instance, regarding a possible health event in subject 4. In certain instances, in may be insightful to perform image recognition on these captured images such as when subject 4 exhibits symptoms of the possible health event in their physical appearance.

Computing device 12 may receive, from IMD 10, a message (e.g., an alert) indicative of a positive detection of the possible health event. Computing device 12 may configure the above hardware/software component to include a confirmation procedure that when executed, generated output data either confirming or rejecting the possible health event as valid. In some examples, the above hardware/software component of computing device 12 confirms an initial positive health event detection by IMD 10 and/or detects a health event independently. Computing device 12 may rely upon the same physiological data used by IMD 10 and/or different physiological parameters for the confirmation. The hardware/software component of computing device 12 may detect a valid health event based on physiological data sensed by sensors of IMB 10 or other sensors, for example, by determining from the sensed physiological data at least one of that movement data satisfies at least one first criterion, at least one of altitude data or location data satisfies at least one second criterion, audio data of the non-human subject satisfies at least one third criterion, or image data of the non-human subject satisfies at least one fourth criterion.

Computing device 12 may be configured for wireless communication with IMB 10. Computing device 12 may retrieve event data and other sensed physiological data from IMD 10 that was collected and stored by the IMD. The present disclosure, in certain places, may use “monitored physiological data” when referring to the sensed physiological data retrieved from IMD 10. In some examples, computing device 12 may be any computing device that can be coupled to subject 4 (e.g., as wearable transponder device) and configured for wireless communication with IMB 10 as a medical device for subject 4. Computing device 12 may communicate with IMD 10 according to the Bluetooth® or Bluetooth® Low Energy (BLE) protocols, as examples. In some examples, computing device 12 is configured for communication with IMD 10, e.g., due to execution of software (e.g., part of a health monitoring application as described herein) enabling communication and interaction with an IMD.

In the example illustrated by FIG. 1, computing device 12 may be a wearable computing device that includes electrodes and other sensors to sense physiological data (e.g., physiological signals) of subject 4. Based on various criterion, computing device 12 may determine satisfaction of the sensed physiological data for a condition triggering upload of at least a portion of such data. Computing device 12 may also collect and store the sensed physiological data to detect health events (e.g., episodes) based on such data. Computing device 12 may be coupled to subject 4 as an animal collar around a neck of subject 4 or may be incorporated into the apparel of subject 4 in a similar manner to human apparel.

Computing device 12 may be configured to communicate with a variety of other devices or systems via a network 16. For example, computing device 12 may be configured to communicate with one or more computing systems, e.g., computing system 20 via network 16. Computing system 20 may be managed by a manufacturer of one or both of IMD 10 and computing device 12 and, for example, provide cloud storage and analysis of collected data, maintenance and software services, or other networked functionality for its respective devices and users thereof. As an option, another computer system may be managed by a manufacturer of computing device 12 (assuming that there are different manufacturers for IMD 10 and computing device 12). Computing system 20 may comprise, or may be implemented by, the Medtronic Carelink™ Network, in some examples. In the example illustrated by FIG. 1, computing system 20 implements HMS 22 as a cloud computing service for remote patient monitoring and health event detection, although in other examples, a plurality of computing systems 20 may implement HMS 22. As will be described in greater detail below, HMS 22 facilities monitoring of subject 4 including detection of health events by system 2, and the responses of system 2 to such health events. HMS 22 may distribute at least some functionality from computing system 20 to device(s) of environment 28.

Computing device 12 may transmit various data, including data retrieved from IMD 10, to computing system 20 via network 16. The data may include sensed data, e.g., values of physiological parameters measured by IMD 10 and, in some cases computing device 12, location data e.g., Global Positioning System (GPS) coordinates provided by satellite 26B, data regarding episodes of arrhythmia or other health events detected by IMD 10 and computing device 12, and other physiological signals or data recorded by IMD 10 and/or computing device 12. HMS 22 may independently retrieve data regarding subject 4 from one or more sources via network 16, such as GPS coordinates of current and/or previous locations of subject 4. HMS 22 may maintain historical data for subject 4 including data regarding historical (e.g., baseline) physiological parameter values, previous health events and treatments, disease states, comorbidities, demographics, height, weight, and body mass index (BMI), as examples, of subjects including subject 4. HMS 22 may use historical data 24 to configure algorithms implemented by IMD 10 and/or computing device 12 to detect health events for subject 4 and any other condition triggering an upload of the above-mentioned data including. In some examples, HMS 22 provides data to computing device 12 and/or IMD 10 for storage therein and use as part of their algorithms for detecting health events. Some of the data provided by HMS 22 includes definitions (e.g., rules) for health events, criteria (e.g., thresholds) for triggering the upload, and, possibly, parameters of a machine learning model.

Network 16 may include one or more computing devices, such as one or more non-edge switches, routers, hubs, gateways, security devices such as firewalls, intrusion detection, and/or intrusion prevention devices, servers, cellular base stations and nodes, wireless access points, bridges, cable modems, application accelerators, or other network devices. Network 16 may include one or more networks administered by service providers and may thus form part of a large-scale public network infrastructure, e.g., the Internet. Network 16 may provide computing devices and systems, such as those illustrated in FIG. 1, access to the Internet, and may provide a communication framework that allows the computing devices and systems to communicate with one another. In some examples, network 16 may include a private network that provides a communication framework that allows the computing devices and systems illustrated in FIG. 1 to communicate with each other but isolates some of the data flows from devices external to the private network for security purposes. In some examples, the communications between the computing devices and systems illustrated in FIG. 1 are encrypted.

As will be described herein, IMD 10 may be configured to detect health events of subject 4 based on data sensed by IMD 10 and, in some cases, other data, such as data sensed by computing device 12 and/or additional sensors in environment 28. In response to detection of a health event, IMD 10 may wirelessly transmit a message to computing device 12. The message may indicate that IMD 10 detected a health event of the subject. The message may indicate a time that IMD 10 detected the health event. The message may include physiological data collected by IMD 10, e.g., data which lead to detection of the health event, data prior to detection of the health event, and/or real-time or more recent data collected after detection of the health event. The physiological data may include values of one or more physiological parameters and/or digitized physiological signals. Examples of health events are a cardiac arrest (e.g., a sudden cardiac arrest), a ventricular fibrillation, a ventricular tachycardia, myocardial infarction, a pause in heart rhythm (asystole), or Pulseless Electrical Activity (PEA), acute respiratory distress syndrome (ARDS), a stroke, a seizure, or a traumatic bodily injury such as a fall.

In response to the message from IMD 10, computing device 12 may output an alarm that may be visual and/or audible, and configured to immediately attract the attention of subject 4 or any person in environment 28 with subject 4, e.g., a caregiver 26. The present disclosure describes alarms as an example of an alert and envisions other example alert types that computing device 12 may generate. Environment 28 may be nature preserve or wildlife refuge, as examples. Computing device 12 may also transmit a message to HMS 22 via network 16. The message may include the data received from IMD 10 and, in some cases, additional data collected by computing device 12 or other devices in response to the detection of the health event by IMD 10. For example, the message may include a location of subject 4 determined by computing device 12. As another example, the message may include input (e.g., vocal responses) from caregiver 26, for example, caregiver 26's responses to queries presented by computing device 12 and/or the other devices.

Other devices in the environment 28 of subject 4 may also be configured to output alarms or take other actions to attract the attention of a caregiver 26, or to otherwise facilitate the delivery of care to subject 4. For example, environment 28 may include one or more Internet of Things (IoT) devices, such as IoT device(s) 30 illustrated in the example of FIG. 1. IoT devices 30 may include, as examples, so called “smart” speakers, cameras, lights, locks, thermostats, appliances, actuators, controllers, or any other smart home (or building) devices. In the example of FIG. 1, IoT device 30C is a smart speaker and/or controller, which may include a display. IoT devices 30 may provide audible and/or visual alarms when configured with output devices to do so. As other examples, IoT devices 30 may cause smart lights throughout environment 28 to flash or blink and unlock doors. In some examples, IoT devices 30 that include cameras, microphones or other sensors may activate those sensors to collect data regarding subject 4, e.g., for evaluation of the condition of subject 4.

Computing device 12 may be configured to wirelessly communicate with IoT devices 30 to cause IoT devices 30 to take the actions described herein. In some examples, HMS 22 communicates with IoT devices 30 via network 16 to cause IoT devices 30 to take the actions described herein, e.g., in response to receiving the alert message from computing device 12 as described above. In some examples, IMD 10 is configured to communicate wirelessly with one or more of IoT devices 30, e.g., in response to detection of a health event when communication with computing devices 12 is unavailable. In such examples, IoT device(s) 30 may be configured to provide some or all of the functionality ascribed to computing devices 12 herein.

Environment 28 includes computing facilities, e.g., a local network 32, by which computing device 12, IoT devices 30, and other devices within environment 28 may communicate via network 16, e.g., with HMS 22. Environment 28 may be configured with a number of networking technologies, such as IEEE 802.11 wireless networks, IEEE 802.15 ZigBee networks, IEEE 802.16 WiMAX wireless broadband networks, a radio access technology (RAT), an ultra-wideband protocol, near-field communication, or the like. In local network 32, for instance, one or more wireless access points, e.g., wireless access points 34A and 34B (collectively, “wireless access points 34”) provide support for wireless (e.g., Wi-Fi) communications throughout environment 28 and enable connectivity with network 16. In some examples, e.g., when local network 32 is unavailable, computing device 12, IoT devices 30, and other devices within environment 28 may be configured to communicate with network 16, e.g., with HMS 22, via a cellular base station 36A and a cellular network. Examples of the cellular network include a 5G NR (New Radio) mobile network, LTE mobile network, a WIMAX protocol network, and/or the like.

As an option, computing device 12, IoT devices 30, and other devices within environment 28 may be configured to communicate with network 16, e.g., with HMS 22, via satellite 36B and a satellite network. Example of the satellite network may include Low Earth Orbit (LEO) networks and Geostationary (GEO) networks. For any of these networks, an integrated transceiver module operative to communicatively couple with satellite 36B may be installed in computing device 12. The integrated transceiver module may be configured to access the satellite network through which computing device 12 transmits sensor data generated by IMD 10 to computing system 20. One example embodiment of the integrated transceiver module may communicatively couple with the Iridium Satellite Constellation, which handles L band (e.g., narrowband) voice and data communications to/from computing device 12.

There also may be examples where the satellite network is combined with another network, such as network 16 (e.g., TCP/IP network) or the cellular network of cellular base station 36A, to establish an enhance wireless connection between computing device 12 and HMS 22 running on computing system 20. Although, in other examples, it may be more efficient to use satellite 36B for only satellite communications (e.g., requesting and receiving GPS coordinates from a GPS server) and Layer 3/Layer 4 network devices for mobile broadband communications.

In the example illustrated in FIG. 1, the above-mentioned types of communication equipment assist computing device 12 in transmitting physiological data to HMS 22. In examples where computing device 12 is a collar-mounted transponder device, the device may implement a technology known as “VHF” which stands for ‘very high frequency” and thus, may be referred to as a pulse collar. The pulse collar may be attached to subject 4 and include VHF transmitter(s) to emit a pulsed radio signal, allowing another device (e.g., camera 6, IoT device(s) 30, and/or the like) to physically locate and possibly, capture additional data regarding subject 4's activities and/or physiology. The other device may employ a VHF receiver and directional antenna. Through this exchange, the other device may track movements of individual animals from a distance.

There are numerous additional advantages to adopting VHF other than as tracking equipment, which is frequently used in combination with satellite tags to upload/download various data including the physiological data of subject 4. Moreover, the present disclosure introduces a new application for computing device 12, which employs VHF to allow for automatic detection of tagged animals. As another benefit, VHF may be used to extend the operating life of a power source (e.g., a battery) within computing device 12, for instance, up to three years. Computing device 12 may employ, as an option, an internal antenna for use when there is a high chance of the antenna being chewed.

To illustrate the power-saving capabilities of VHF, computing device 12 may operate in a low-power/power-off mode except for when computing device 12 transitions into a second power mode during which computing device 12 determines an availability of a communication network within environment 28. Computing device 12 may be programmed to wake up (e.g., on a schedule or at regular intervals such as every five minutes) for a limited amount of time to poll a surrounding area within environment 28. Polling refers to a mechanism by which two devices may negotiate a stable connection; for example, computing device 12 may search for a device having a VHF receiver, such as camera 6, IoT device(s) 30, or wireless access point(s) 34, by broadcasting polling messages and listening for any acknowledgement messages. Computing device 12 may be configured to wake up for at least a sufficient amount of time to thorough search environment 28 for the device having the VHF receiver and if no such device is located, computing device 12 may be programmed to return to the power-off mode or the low-power mode. If such a device is detected and a connection can be made, computing device 12 may transmit its stored data including the physiological data of subject 4. The transmission may include data identified in an information request from caregiver 26. The transmission may concord with a schedule set by caregiver 26.

Computing device 12, and in some examples IoT devices 30, may include input devices and interfaces to allow caregiver 26 to override the alarm in the event the detection of the health event by IMD 10 was false. In some examples, one or more of computing device 12 and IoT device(s) 30 may implement an event assistant. The event assistant may provide a conversational interface for subject 4 and/or caregiver 26 to exchange information with the computing device or IoT device. The event assistant may query the user regarding the condition of subject 4 in response to receiving the alert message from IMD 10. Responses from the user may be used to confirm or override detection of the health event by IMD 10, or to provide additional information about the health event or the condition of subject 4 more generally that may improve the efficacy of the treatment of subject 4. For example, information received by the event assistant may be used to provide an indication of severity or type (differential diagnosis) for the health event. The event assistant may use natural language processing and context data to interpret utterances by caregiver 26 or another human user. In some examples, in addition to receiving responses to queries posed by the assistant, the event assistant may be configured to respond to queries posed by the user. The input devices and/or interfaces of computing device 12 and in some examples, IoT devices 30 may process other forms of input besides queries. Caregiver 26 or another human user may submit information requests to computing device 12 for that device to respond by retrieving the requested data from IMD 10 and communicating, as output, the requested data via network 16 to HMS 22.

In some examples, computing device 12 and/or HMS 22 may implement one or more algorithms to evaluate the sensed physiological data received from IMD 10, and in some cases additional physiological or other data sensed or otherwise collected by the computing device(s) or IoT devices 30, to confirm or override the detection of the health event by IMD 10. In some examples, computing device 12 and/or computing system 20 may have greater processing capacity than IMD 10, enabling more complex analysis of the data. In some examples, the computing device 12 and/or HMS 22 may apply the data to a machine learning model or other artificial intelligence developed algorithm, e.g., to determine whether the data is sufficiently indicative of the health event.

In examples in which computing device 12 are configured to perform a health event confirmation analysis, computing device 12 may transmit alert messages to HMS 22 and/or IoT device(s) 30 in response to confirming the health event. In some examples, computing device 12 may be configured to transmit the alert messages prior to completing the confirmation analysis, and transmit cancellation messages in response to the analysis overriding the detection of the health event by IMD 10. HMS 22 may be configured to perform a number of operations in response to receiving an alert message from computing device 12 and/or IoT device(s) 30. HMS 22 may be configured to cancel such operations in response to receiving a cancellation message from computing device 12 and/or IoT device(s) 30.

For example, HMS 22 may be configured to transmit alert messages to one or more computing devices 38 associated with one or more care providers 40 via network 16. As described herein, a legitimate detection of a health event by IMD 10 or computing device 12 may prompt HMS 22 to automatically transmit an alert message to each of the one or more computing devices 38. HMS 22 may receive a notification—which also may constitute an alert message—of the detected health event, causing the automatic transmission of the above alert message(s). Similarly, HMS 22 may be configured to transmit alert messages to other computing devices, such as a computing device of a human who is not amongst care provider(s) 40.

As another example, HMS 22 may be configured to interact with a human via their user device, which may be user device 42 or computing device 12 of FIG. 1. This interaction may follow an alert message transmission or, alternatively, may not involve any alert message. Care providers 40 may maintain educational and training resources for HMS 22 to distribute, for example, if needed, by a caregiver (e.g., caregiver 26 of FIG. 1) or a bystander in view of a health event occurring in a non-human subject. HMS 22 may communicate to user device 42 various information to be presented on a user interface for the caregiver to review. Examples of this information include guidance (e.g., instructions) for sedating animals for procedures, performing exams, giving vaccinations, taking blood samples, administering fluids, performing surgeries when needed, prescribing medications, evaluating and treating wounds, taking x-rays and ultrasounds, cleaning teeth, assisting with captive breeding programs, providing intensive care for very young animals abandoned by their parents, and other duties of a wildlife veterinarian.

Care providers 40 may include various organizations consisting mainly of animal health professionals (e.g., veterinary surgeons/physicians) and/or those who provide emergency services and/or non-emergency medical care to animals and other non-human subjects (e.g., subject 4 of FIG. 1). Animal health professionals may specialize in treating many different types of wildlife, including birds, amphibians, reptiles, and mammals. These practitioners may receive training and learning materials from care providers 40 operating educational institutions (e.g., colleges for veterinary medicine), medical centers (e.g., animal hospitals and veterinary offices), emergency services for wildlife and/or other animals (e.g., animal emergency services/urgent care, veterinary emergency clinics, wildlife emergency and conflict services, and wildlife rescue/rehabilitators at a rehabilitation facility), and/or research institutions (e.g., clinical research for enhancing veterinary medicine).

Computing devices 38 may include smartphones, desktop, laptop, or tablet computers, or workstations associated with such systems or entities, or employees of such systems or entities. The alert messages may include any of the data collected by IMD 10, computing device 12, and IoT device(s) 30, including sensed physiological data, time of the health event, location of subject 4, and results of the analysis by IMD 10, computing device 12, IoT device(s) 30, and/or HMS 22. The information transmitted from HMS 22 to care providers 40 may improve the timeliness and effectiveness of treatment of the health event of subject 4 by care providers 40. In some examples, instead of or in addition to HMS 22 providing an alert message to one or more computing devices 38 associated with an EMS care provider 40, computing device 12 and/or IoT devices 30 may be configured to automatically contact EMS, e.g., autodial 911, in response to receiving an alert message from IMD 10. Again, such operations may be cancelled by subject 4, caregiver 26, or another user via a user interface of computing device 12 or IoT device(s) 30, or automatically cancelled by computing device 12 based on a confirmatory analysis performed by the computing device(s) overriding the detection of the health event by IMD 10.

Similarly, HMS 22 may be configured to transmit an alert message to computing device 42 (e.g., a user device) of caregiver 26, which may improve the timeliness and effectiveness of treatment of the health event of subject 4 by caregiver 26. Computing device 42 may be similar to computing devices 12 and computing devices 38, e.g., a smartphone. In some examples, HMS 22 may determine that caregiver 26 is proximate to subject 4 based on a location of subject 4, e.g., received from computing device 12, and a location of computing device 42, e.g., reported to HMS 22 by an application implemented on computing device 42. In some examples, HMS 22 may transmit the alert message to any computing devices 42 in an alert area determined based on the location of subject 4, e.g., by transmitting the alert message to all computing devices in communication with base station 36A.

In some examples, the alert message to caregiver 26 may be configured to assist a human layperson (e.g., a bystander, a park ranger, or an animal health professional such as a biologger, a field researcher, and/or the like) in treating subject 4. For example, the alert message to caregiver 26 may include a location (and in some cases a description) of subject 4, the general nature of the health event, directions for providing care to subject 4, such as directions for providing cardio-pulmonary resuscitation (CPR) or other treatment, a location of nearby medical equipment for treatment of subject 4 and instructions for use of the equipment. In some examples, computing device 12, IoT device(s) 30, and/or computing device 42 may implement an event assistant configured to use natural language processing and context data to provide a conversational interface for bystander 42. The assistant may provide caregiver 26 with directions for providing care to subject 4 and respond to queries from caregiver 26 about how to provide care to subject 4.

In some examples, HMS 22 may mediate bi-directional audio (and in some cases video) communication between care providers 40 and subject 4 or caregiver 26. Such communication may allow care providers 40 to evaluate the condition of subject 4, e.g., through communication with subject 4 or caregiver 26, or through use of a camera or other sensors of the computing device or IoT device, in advance of the time they will begin caring for the subject, which may improve the efficacy of care delivered to the subject. Such communication may also allow the care providers to instruct bystander 42 regarding first responder treatment of subject 4.

In some examples, HMS 22 may control dispatch of a drone 46 to environment 28, or a location near environment 28 or subject 4. Drone 46 may be a robotic device, such as unmanned aerial vehicle (UAV) or another robot. Drone 46 may be equipped with a number of sensors and/or actuators to perform a number of operations. For example, drone 46 may include a camera or other sensors to navigate to its intended location, identify subject 4 and, in some cases, caregiver 26, and to evaluate a condition of subject. In some examples, drone 46 may include user interface devices to communicate with subject 4 and/or caregiver 26. In some examples, drone 46 may provide directions to caregiver 26, to the location of subject 4 and regarding how to provide first responder care, such as CPR, to subject 4. In some examples, drone 46 may carry medical equipment, e.g., an AED, splints, bandages, and/or medication to the location of subject 4.

In some examples, HMS 22 may control dispatch of drone 46 to a location (e.g., a habitat such as a den or nest) of subject 4. If emergency care is needed, drone 46 may be equipped to perform certain medical procedures. For example, drone 46 may be configured to access an airway and start ventilation. As another example, drone 46 may move subject 4 away from harm (e.g., by moving subject away a fire (e.g., forest fire) or an electrical hazard and/or facilitating caregiver access to subject 4). HMS 22 may program the robotic device to deliver therapy or recommend therapy based on a cohort analysis of subject 4's current disease state and sensed physiological data. Drone 46 may also be operative to put the subject into a hypothermic state, if needed. In some instances, drone 46 may confirm whether there are any caregivers proximate to subject 4 and then, activate normal operation if none are detected. Drone 46 may be configured to summon a caregiver to support therapy delivery or other medical care for subject 4. Drone 46 may be configured to make an ECG or pulse measurement or may operate as an AED by touching two parts of subject 4's body with extendable arms having electrodes.

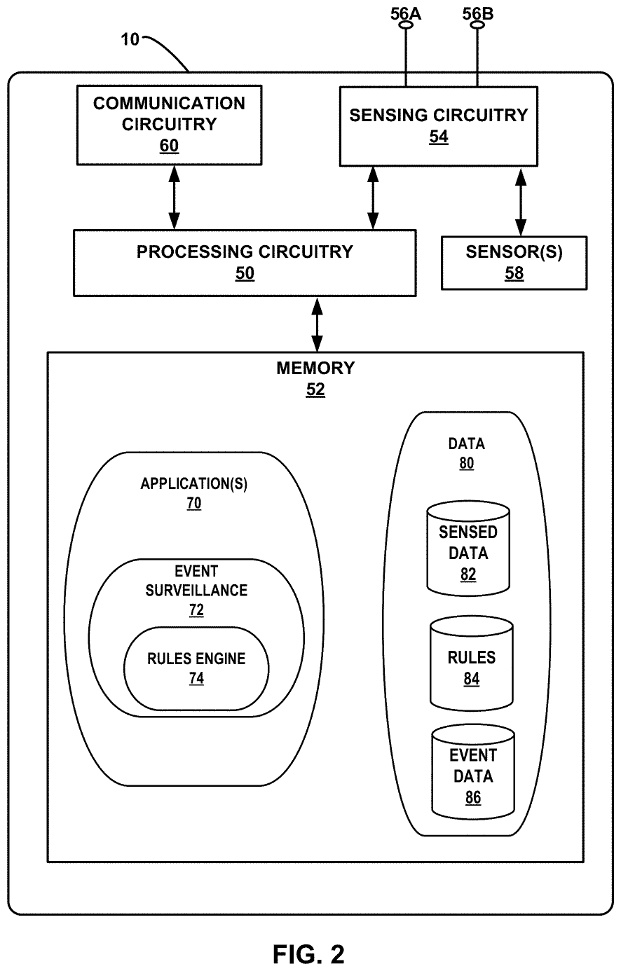

FIG. 2 is a block diagram illustrating an example configuration of IMD 10 of FIG. 1. As shown in FIG. 2, IMD 10 includes processing circuitry 50, memory 52, sensing circuitry 54 coupled to electrodes 56A and 56B (hereinafter, “electrodes 56”) and one or more sensor(s) 58, and communication circuitry 60.

Processing circuitry 50 may include fixed function circuitry and/or programmable processing circuitry. Processing circuitry 50 may include any one or more of a microprocessor, a controller, a graphics processing unit (GPU), a tensor processing unit (TPU), a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more GPUs, one or more TPUs, one or more DSPs, one or more ASICs, or one or more FPGAs, as well as other discrete or integrated logic circuitry. The functions attributed to processing circuitry 50 herein may be embodied as software, firmware, hardware, or any combination thereof. In some examples, memory 53 includes computer-readable instructions that, when executed by processing circuitry 50, cause IMD 10 and processing circuitry 50 to perform various functions attributed herein to IMD 10 and processing circuitry 50. Memory 53 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Sensing circuitry 54 may monitor signals from electrodes 56 in order to, for example, monitor electrical activity of a heart of subject 4 and produce ECG data for subject 4. In some examples, processing circuitry 50 may identify features of the sensed ECG, such as heart rate, heart rate variability, intra-beat intervals, and/or ECG morphologic features, to detect an episode of cardiac arrhythmia or other changes in health of subject 4. Processing circuitry 50 may store the digitized ECG and features of the ECG used to detect the episode in memory 52 as episode data for the detected arrhythmia episode.

In some examples, sensing circuitry 54 measures impedance, e.g., of tissue proximate to IMD 10, via electrodes 56. The measured impedance may vary based on respiration and a degree of perfusion or edema. Processing circuitry 50 may determine physiological data relating to respiration, perfusion, and/or edema based on the measured impedance.

In some examples, IMD 10 includes one or more sensors 58, such as one or more accelerometers, microphones, optical sensors, temperature sensors, and/or pressure sensors. In some examples, sensing circuitry 52 may include one or more filters and amplifiers for filtering and amplifying signals received from one or more of electrodes 56 and/or sensors 58. In some examples, sensing circuitry 54 and/or processing circuitry 50 may include a rectifier, filter and/or amplifier, a sense amplifier, comparator, and/or analog-to-digital converter. Processing circuitry 50 may determine physiological data, e.g., values of physiological parameters of subject 4, based on signals from sensors 58, which may be stored in memory 52.

Memory 52 may store applications 70 executable by processing circuitry 50, and data 80. Applications 70 may include an event surveillance application 72. Processing circuitry 50 may execute event surveillance application 72 to monitor subject 4 for (e.g., sufficient evidence) of one or more conditions triggering an upload of the at least a portion of data 80. In some examples, processing circuitry 50 may execute event surveillance application 72 to detect a health event of subject 4 based on combination of one or more of the types of physiological data described herein, which may be stored as sensed data 82. In some examples, sensed data 82 may additionally include data sensed by other devices, e.g., computing device 12, and received via communication circuitry 60. Event surveillance application 72 may be configured with rules engine 74. Rules engine 74 may apply rules 84 to sensed data 82 (e.g., to perform the health event detection). Rules 84 may include one or more models, algorithms, decision trees, and/or thresholds. In some cases, rules 84 may be developed based on machine learning.

In other cases, rules 84 may be designed to limit resource consumption and/or conserve resource capacities and/or capabilities. Rules 84 may include a rule directing transmissions of the physiological data of subject 4 to the collar-mounted transponder device for subsequent transmission to HMS 22 where the physiological data may be stored as historical data for subject 4. In one example, rules 84 may include a schedule where IMD 10 uploads data to a VHF receiver according to the IMD 10's own wake-up schedule. An example schedule may be set to every five minutes. Another example rule 84 may define a length of each interval in the wake-up schedule.

Event surveillance 72 may be operative to automatically adjust the conditions triggering an automatic or imminent upload, for example, by calibrating certain criterion, thresholds, settings, parameters, and/or the like for health event detection (e.g., cardiac episode detection thresholds) in view of historical data 24 for subject 4. For example, rules engine 74 may set a detection rate to a higher or lower heart rate with a sliding scale, thereby enabling more accurate health event detection. Rules engine 74 may employ machine learning techniques to learn appropriate settings for rules 84 as well as how to identify outliers (e.g., greater than three (3) standard deviations in a normal distribution). Rules engine 74 may differentiate between non-human subject 4 and other species with respect to rules 84 such that IMD 10 may be programmed differently if used for the other species.

Amongst sensors 58, IMD 10 may employ multi-temperature sensors to get a thermopile to measure a heat flux in terms of magnitude and direction (e.g., into and/or out of subject 4). IMD 10 may program into logic for rules engine 74 one or more criterion configured to determine whether subject 4 is in a warm environment or if their body temperature is increasing. Sensors 58 may include an acceleration detector that can activate long-term ECG/EGM storage. Sensors 58 may include an acoustic sensor for evaluating heart sounds and respiration. Sensors 58 may include an infrared sensor to detect blood flow within subject 4.

Rules engine 74 may implement example anti-poaching functionality where rules 84 include criteria that are configured to evaluate various physiological parameter values. Determining satisfaction of the criteria causes rules engine 74 to prompt an alert mechanism of event surveillance 72. Rules engine 74 may apply the example criteria to measurements of temperature, heart rate, acceleration, and/or impedance to determine whether subject 4 is being poached. According to one example where the example criteria are satisfied (e.g., because subject 4 has been caught by a poacher), rules engine 74 determines that subject 4 has a high heart rate and has recorded little or no motion in a recent past. In another examples, rules engine 74 may rely upon velocity or altitude sensors to determine poaching. In yet another example, long term recording at IMD 10 or computing system 20 may be triggered when subject 4 is determined to have a high heart rate with little or no motion over the recent past.

Shiver detection rules of rules 84 may be employed by rules engine 74 to look for and filter muscle noise from sensed data 82. Subject 4 is subject to shiver for a variety of reasons, causing IMD 10 to move (e.g., accelerate) at an above or high rate (e.g., of change). One example shiver detection rule may establish a threshold velocity/acceleration rate indicative of a shivering subject. If subject 4 is causing IMD 10 to move (e.g., accelerate) at a rate meeting or exceeding the threshold, subject 4 most likely is shivering. A positive detection of shivering may prompt IMD 10 to change filtering or gain. In one example set of shiver detection rules, IMD 10 may change the digital filtering characteristics and/or the gain of an amplification being applied to physiological signals. For example, a signal having substantial noise or a significant “shiver” or other movement by subject 4 may cause saturation of a measurement circuit and/or go out of range in some way.

One or more rules of rules 84 may dynamically control the automatic transmission of at least a portion of sensed data 82 and/or event data 86 to computing device 12. For example, the one or more rules may define different modes for the automatic data transmission. Upon determining satisfaction of one or more conditions triggering the automatic transmission, event surveillance 72 may communicate data at a pre-determined rate set by rules 84 (assuming that IMD 10 is communicatively coupled via a wireless connection to computing device 12). Rules engine 74 may determine a new transfer rate for the transmission of the at least a portion of sensed data 82 based on the connectivity with computing device 12. Accordingly, if IMD 10 cannot communicate with computing device 12 (e.g., because IMD 10 lost the wireless connection with computing device 12), IMD 10 may continue to transfer data but at a different (e.g., lower) rate.

Another condition triggering the automatic transmission may be satisfied when IMD 10 receives from computing device 12 an initiation of an action (e.g., to perform data collection and transmission). Rules 84 may define different modes for the wideband data collection where sensed data from sensors 58 is retrieved and then, prepared for the automatic transmission to computing device 12. If IMD 10 determines that the wireless connection is lost and it cannot communicate with computing device 12, IMD 10 may continue to collect data from sensors 58 but at a different density.

Another condition triggering the automatic transmission may be satisfied if there is a delta temperature across IMD 10. In such a case, IMD 10 invokes a transmission mode indicative of higher density recording.

Another condition whose satisfaction triggers the automatic transmission may be insufficient resources in IMD 10. Networking technology in IMD 10 may enable streaming of the sensed data (e.g., as either wide-band signals or wide-band episode data) to an external device, such as computing device 12. As the sensed data is communicated, memory previously allocated for storing the sensed data is freed and made available for new data. In accordance with another rule of rules 84, when available memory space in IMD 10 falls below a certain threshold (e.g., minimum), rules engine 84 prompts event surveillance 72 to commence the data transmission to computing device 12. Hence, having less than a sufficient capacity of available memory is one example way of satisfying the above condition triggering the automatic transmission of data to computing device 12.

As described herein, IMD 10 may be wirelessly connected to one or more external devices including one or more computing devices and/or one or more external sensors. An external device, such as computing device 12, may be configured to wirelessly recharge (e.g., a battery of) IMD 10. IMD 10 may employ an external infrared sensor (e.g., ground sensor 8 of FIG. 1) to determine blood flow measurements. IMD 10 may use a watch mechanism for charging its battery by way of some wearable device that could provide energy through the skin of subject 4.

In addition to being an implanted medical device, IMD 10 may be bioresorbable or include one or more bioresorbable components. As examples, event surveillance application 72 may detect a cardiac arrest, a ventricular fibrillation, a ventricular tachycardia, a cardiac pause of asystole, pulseless electrical activity (PEA), or a myocardial infarction based on an ECG and/or other physiological data indicating the electrical or mechanical activity of heart of subject 4 (FIG. 1). In some examples, event surveillance application 72 may detect stroke based on such cardiac activity data. In some examples, sensing circuitry 54 may detect brain activity data, e.g., an electroencephalogram (EEG) via electrodes 56, and event surveillance application 72 may detect stroke or a seizure based on the brain activity alone, or in combination with cardiac activity data or other physiological data. In some examples, event surveillance application 72 detects whether the subject has fallen based on data from an accelerometer alone, or in combination with other physiological data. When event surveillance application 72 detects a health event, event surveillance application 72 may store the sensed data 82 that lead to the detection (and in some cases a window of data preceding and/or following the detection) as event data 86.

In some examples, in response to detection of a health event, processing circuitry 50 transmits, via communication circuitry 60, event data 86 for the event to computing device 12 (FIG. 1). This transmission may be included in a message indicating the health event, as described herein. Processing circuitry 50 may package into this message transmission a number of informational attributes including dataset(s) of (e.g., contemporaneous) location information (e.g., GPS coordinates), datasets of physiological parameter values, and other datasets. Transmission of the message may occur on an ad hoc basis and as quickly as possible. Communication circuitry 60 may include any suitable hardware, firmware, software, or any combination thereof for wirelessly communicating with another device, such as computing devices 12 and/or IoT devices 30.

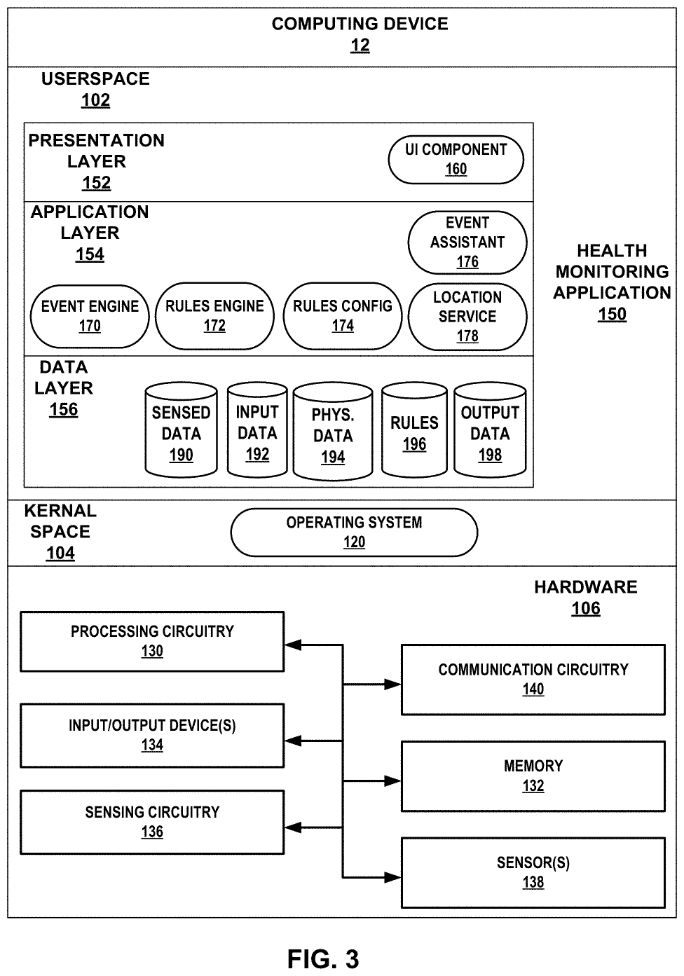

FIG. 3 is a block diagram illustrating an example configuration of a computing device of subject 4, which may correspond to computing device 12 illustrated in FIG. 1. In some examples, computing device 12 takes the form of a wearable computing device with a transponder. In some examples, IoT device 30 may be configured similarly to the configuration of computing device 12 illustrated in FIG. 3.

As shown in the example of FIG. 3, computing device 12 may be logically divided into user space 102, kernel space 104, and hardware 106. Hardware 106 may include one or more hardware components that provide an operating environment for components executing in user space 102 and kernel space 104. User space 102 and kernel space 104 may represent different sections or segmentations of memory, where kernel space 104 provides higher privileges to processes and threads than user space 102. For instance, kernel space 104 may include operating system 120, which operates with higher privileges than components executing in user space 102.

As shown in FIG. 3, hardware 106 includes processing circuitry 130, memory 132, one or more input devices 134, one or more output devices 136, one or more sensors 138, and communication circuitry 140. Although shown in FIG. 3 as a stand-alone device for purposes of example, computing device 12 may be any component or system that includes processing circuitry or other suitable computing environment for executing software instructions and, for example, need not necessarily include one or more elements shown in FIG. 3.

Processing circuitry 130 is configured to implement functionality and/or process instructions for execution within computing device 12. For example, processing circuitry 130 may be configured to receive and process instructions stored in memory 132 that provide functionality of components included in kernel space 104 and user space 102 to perform one or more operations in accordance with techniques of this disclosure. Examples of processing circuitry 130 may include, any one or more microprocessors, controllers, GPUs, TPUs, DSPs, ASICs, FPGAs, or equivalent discrete or integrated logic circuitry.

Memory 132 may be configured to store information within computing device 12, for processing during operation of computing device 12. Memory 132, in some examples, is described as a computer-readable storage medium. In some examples, memory 132 includes a temporary memory or a volatile memory. Examples of volatile memories include random-access memories (RAM), dynamic random-access memories (DRAM), Ferroelectric random-access memories (FRAM), static random-access memories (SRAM), and other forms of volatile memories known in the art. Memory 132, in some examples, also includes one or more memories configured for long-term storage of information, e.g., including non-volatile storage elements. Examples of such non-volatile storage elements include magnetic hard discs, optical discs, floppy discs, flash memories, or forms of electrically programmable memories (EPROM) or electrically erasable and programmable (EEPROM) memories.

One or more input/output devices 134 of computing device 12 may receive input, e.g., from a human user such as a bystander or a caregiver of subject 4. Examples of input are tactile, audio, kinetic, and optical input. Input/output devices 134 may include, as examples, a mouse, keyboard, voice responsive system, camera, buttons, control pad, microphone, presence-sensitive or touch-sensitive component (e.g., screen), or any other device for detecting input from the human user or a machine. Input/output devices 134 of computing device 12 may generate output, e.g., to the human user. Examples of output are tactile, audio, and visual output. Output devices 134 of computing device 12 may include a presence-sensitive screen, sound card, video graphics adapter card, speaker, cathode ray tube (CRT) monitor, liquid crystal display (LCD), light emitting diodes (LEDs), or any type of device for generating tactile, audio, and/or visual output.

Sensing circuitry 136 may monitor/capture sensor output (signals) of one or more sensors 138 and then, generate sensed data 190 from captured sensor signals that encode sensed physiological data as described herein. In some examples, sensing circuitry 136 may include one or more filters and amplifiers for filtering and amplifying signals received from sensors 138. Sensed data 190 may include raw sensor output from which processing circuitry 130 may generate refined (e.g., parameterized) physiological data 194, e.g., values of physiological parameters of subject 4, which may be stored in memory 132 and then, uploaded to the remote computing system. In some examples, physiological data 194 is uploaded to an external device (e.g., network device) for storage as historical data and to facilitate (e.g., remote) health event monitoring (e.g., by computing system 20 of FIG. 1) for changes in subject 4's (e.g., cardiac) health.

One or more sensors 138 of computing device 12 may sense physiological parameters or signals of subject 4. Sensor(s) 138 may include electrodes, 3-axis accelerometers, an optical sensor, an impedance sensor, a temperature sensor, a pressure sensor, a heart sound sensor, and other sensors, and sensing circuitry (e.g., including an ADC), similar to those described above with respect to IMD 10 and FIG. 2.

Communication circuitry 140 of computing device 12 may communicate with other devices by transmitting and receiving data. Communication circuitry 140 may include a network interface card, such as an Ethernet card, an optical transceiver, a radio frequency transceiver, or any other type of device that can send and receive information. For example, communication circuitry 140 may include a radio transceiver configured for communication according to standards or protocols, such as 3G, 4G, 5G, Wi-Fi (e.g., 802.11 or 802.15 ZigBee), Bluetooth®, or Bluetooth® Low Energy (BLE).

In one example, communication circuitry 140 includes a transmitter configured to broadcast pulsed signals in the VHF portion of the electromagnetic spectrum (30 to 300 MHz). Study animals like subject 4 are assigned unique frequencies, so that individual animals can be followed. Researchers may use specialized antennas and receivers to track these individual animals. VHF may be used in combination with satellite tags to download data, thereby enabling automatic detection of tagged animals. VHF transceivers may be used for a wide variety of animals and extend the operating life of the battery can last up to three years. VHF transceivers are easily affixed to a (neck) collar or a (body) harness. There may also be an internal antenna for use when there is a high chance of an external antenna being chewed.

As shown in FIG. 3, health monitoring application 150 executes in user space 102 of computing device 12. Health monitoring application 150 may be logically divided into presentation layer 152, application layer 154, and data layer 156. Presentation layer 152 may include a user interface (UI) component 160, which generates and renders user interfaces of health monitoring application 150. Subject 4 as described herein is a non-human subject such as a wild animal and, most likely, would not use the user interfaces for their intended purpose; for at least that reason, UI component may generate a specific user interface through which the non-human subject 4 interacts with (e.g., navigates through) information and functionality of the health monitoring application.

Application layer 154 may include, but is not limited to, an event engine 170, rules engine 172, rules configuration component 174, event assistant 176, and location service 178. Event engine 172 may be responsive to receipt of an alert transmission from IMD 10 indicating that IMD 10 detected a health event. Event engine 172 may control performance of any of the operations in response to detection of a health event as described herein, such as activating an alarm, transmitting alert messages to HMS 22, controlling IoT devices 30, and analyzing data to confirm or override the detection of the health event by IMD 10. Event engine 172 may decide to suppress any one of the alert messages from IMD 10 and then, inform IMD 10 or HMS 22 by way of sending an alert cancellation (message). Event engine 172 also may relay an alert cancelation from IMD 10 to HMS 22 and vice versa.

In general, event engine 172 evaluates conditions triggering an upload of data to a remote computing system. A schedule (e.g., time of day or week), network connectivity (or lack thereof), are insufficient resources (e.g., memory) are among the examples of possible conditions triggering the upload. Detecting a health event as described herein satisfies at least one example condition triggering the upload. In some examples, event engine 172 may determine that the health event has occurred, is occurring, or will occur based on evidence found in sensed data 190 and/or physiological data 194. This may be in response to receiving, from IMD 10, input data 192 indicative of a positive detection of the health event. Input data 192 may include physiological parameter values relied upon for the detection. Event engine 172 may be configured to store those values in physiological data 194. Alternatively, event engine 172 may detect the health event without first receiving the alert message from IMD. After detecting the health event according to one alternative example, event engine 172 invokes the alert mechanism to package the detected health event into an alert message to be communicated to the remote computing system.

Input/output devices 134 and/or sensors 138 may include an acoustic microphone to capture voice/audio input. The acoustic microphone may also provide non-contact monitoring of certain physiological parameters, such as heart rate, respiration rate, and/or the like. Processing circuitry 130 may be configured to apply speech recognition functionality to the captured voice/audio input and then, based on the recognized speech data, determine whether a condition has been satisfied, triggering an upload (e.g., an automatic data recordation/collection/transmission).

In some examples, computing device 12 may be a wearable device having processing circuitry 130 e.g., via health monitoring application 150, to provide health event monitoring and detection. Processing circuitry 130 of computing device 12 may receive a message indicating detection of health event by IMD 10. In some examples, computing device 12 generates an alert, for instance, by activating an audible or visual alarm. Processing circuitry 130 of computing device 12 may analyze the subject 4's sensed physiological data, for example, to confirm or reject the detection of the health event and the alert. Processing circuitry 130 of computing device 12 may execute instructions to determine whether to confirm the health event based on the analysis, including caregiver 26 responses to queries. As described herein, processing circuitry 130 may employ a rules engine 172 to render a determination (e.g., a likelihood) regarding whether the sensed physiological data includes sufficient evidence for the health event.

Processing circuitry 130 may be further configured to run anti-poaching functionality based on data provided by input/output devices 134 and/or sensors 138. In some examples, velocity sensors and/or altitude sensors of sensors 138 provide a current velocity and/or a current altitude for subject 4, and if one or both of these current measurements indicate that subject 4 is in an unfamiliar or dangerous environment and/or being pursued by poachers, processing circuitry 130 executes the alert mechanism and other functionality enabled by event engine 172 and in support of anti-poaching.

Another example condition for which satisfaction triggers the upload of relevant information and/or an alert message may include multiple criteria or a single criterion for evaluating acceleration data of IMD 10 and/or computing device 12. An example criterion may establish a threshold (e.g., minimum/maximum) rate for the current acceleration measurement such that when the current acceleration measurement satisfies this threshold (e.g., by exceeding the minimum and being below the maximum), event engine 172 initiates an action causing IMD 10 to monitor/collect physiological data for automatic transmission to computing device 12 where the monitored physiological data is combined with sensed data 190 and/or (sensed) physiological data 194 and then, uploaded (via a wireless connection) to the remote computing system. The same criterion or a different criterion, if satisfied by one or more acceleration measurements, may prompt event engine 172 to communicate a relevant alert message to the remote computing system without triggering an upload of at least a portion of physiological data 194.

Another example condition for which satisfaction triggers an upload of monitoring/sensed physiological information for subject 4 is detection of a waypoint of a communication network. Such a detection includes a determination that the waypoint is a network device that is available for transmitting (e.g., relaying) data to the remote computing system. In response to the detection, computing device 12 may retrieve, via communication circuitry 140, at least a portion of the monitored physiological data from the medical device and then, proceed to transmit the retrieved portion until completion.

There are a number of mechanisms for determining availability of the communication network (e.g., a satellite network, a cellular network, and/or the like) to the remote computing system. In one example, processing circuitry 130 may be configured to broadcast a polling message for detecting a network device operating as a waypoint of the communication network for the remote monitoring system. The network device may be located within an area (e.g., environment 28) of computing device 12. In another example, processing circuitry 130 may be configured to listen for a second polling message or an acknowledgment of the first polling message for detecting the network device of the communication network within the area. In another example, processing circuitry 130 may be configured to determine whether location data indicates the network device (e.g., station) of the communication network is within an effective distance for receiving a data transmission based on receiving at least one of the second polling message or an acknowledgment of the first polling message. The location data may be provided in either the second polling message or the acknowledgment of the first polling message. The location data may be facilitated through employment of VHF technology as described herein.

Some example conditions for which satisfaction triggers an upload of monitoring/sensed physiological information for subject 4 may be designed to limit resource consumption and/or conserve resource capacities and/or capabilities. In one example, caregiver 26 may configure settings of computing device 12 to operate in one of multiple power modes. One power mode may be a power-off or low-power mode where computing device 12 conserves as much resource capacities as possible. Caregiver 26 may further configure settings of computing device 12 to follow an operating schedule where computing device 12 uploads data to a VHF receiver by transitioning from to a second power mode that consumes more power than the power-off/low-power mode. An example schedule may be set to every five minutes. This schedule functions as a condition triggering the upload, assuming there is an available network device to relay the transmitted data. The schedule may indirectly cause computing device 12 to download data stored in IMD 10, assuming such data is available, in preparation for the data transmission.

Another example condition for which satisfaction triggers an upload of monitoring/sensed physiological information for subject 4 may be reception of an information request from caregiver 26. In response, processing circuitry 130 of computing device 12 may be configured to retrieve at least a portion of the monitored physiological data in accordance with the information request received from the remote monitoring system. The information request may specify certain datasets to upload over other datasets.

Computing device 12 may be configured with a hardware resource, such as memory 132, to store a number of datasets for subject 4. Processing circuitry 130, in accordance with a schedule, may be operative to automatically transmit, via communication circuitry 140, one or more of these datasets to the remote computing system where such datasets are maintained (e.g., archived) as historical data; however, if memory 132 is determined to have an insufficient amount (e.g., capacity) of available space to store any more datasets, processing circuitry 130 of computing device 12 may proceed with an imminent transmission of the stored datasets to free additional memory space. Once a dataset has been transferred to the remote computing system, computing device 12 may overwrite the previously allocated memory space for that dataset, for example, with NULL data or by storing a new dataset in that memory space.

Example system 2 may configure IMD 10 and computing device 12 to implement an architecture for resources (e.g., storage/memory resources) in which datasets of physiological parameters are arranged and then, uploaded to the remote computing system without a substantial latency in between storage and transmission. The architecture may implement a scheme or file system for organizing the datasets in a manner that facilitates their immediate retrieval from memory and subsequent transmission via networking resources. These datasets may be placed in a memory buffer in preparation of the transmission or, as an alternative, undergo an automatic transmission. Instead of having a substantial amount of storage resources for buffering and then, transmitting the dataset, IMD 10 and computing device 12 may be configured with fewer resources and facilitate a low latency between buffering and (subsequent) transmission. This may be because the architecture enables IMD 10 and computing device 12 to make efficient use of their memory and other storage resources.

Computing device 12, in some alternatives, may have substantial resource capacities and therefore, may store new data for subject 4 until a next transmission time (which may set according to a scheduled or ad-hoc). Computing device 12 may be configured with a hardware resource, such as memory 132, whose capacity may be measured in terabytes. To ensure each overwritten dataset is successfully transferred to the remote computing system, computing device 12 may be equipped with a substantial storage capacity. Given the substantial capacity, memory 132 may function as an external storage resource for IMD 10 (possibly) to use in off-loading datasets of physiological data for subject 4 in addition to its role as internal local storage for computing device 12.

Computing device 12, while attached to subject 4, may store a number of datasets for subject 4 including physiological data transmitted by IMD 10. Some of the datasets may include data that has been overwritten at IMD 10 and therefore, the only copy of that information. As subject 4 moves around an area (e.g., environment 28), computing device 12 detects network connectivity by sensing a waypoint (e.g., access point(s) 34A-34B or base station 36A) to network 16) that is located within a certain proximity (e.g., among other criteria). Having the waypoint with the certain proximity allows computing device 12 to successfully transfer the physiological data generated by IMD 10. Upon/after sensing the waypoint, computing device 12 may commence transmitting each of the datasets in memory 132. In this manner, subject 4's movement/position may trigger an automatic data transmission by computing device 12.

As indicated herein, IMD 10 may first transmit its stored data and then, perform an overwrite of corresponding memory sectors. After transmitting a dataset, IMD 10 typically overwrites the memory sectors for that dataset, for example, with NULL data or by storing a new dataset in that memory space. IMD 10 may benefit from the substantial capacity of the available memory space in computing device 12, for example, by using that memory space as an external storage resource. In this manner, IMD 10 may be programmed to (e.g., automatically) transfer physiological data of subject 4 to computing device 12 for storage, thereby allocating only a fraction (or none, if possible) of a local storage resource in IMD 10 for storing the same physiological data of subject 4. To mitigate incidents of data loss, IMD 10 may benefit from the substantial capacity of memory 132 by sending wideband the physiological data of subject 4 to computing device 12 for buffering and/or transmission. Given that the systems and techniques described herein are compatible with different networking and/or storage technologies, embodiments of IMD 10 and computing device 12 may be adapted to the implications of implementing those technologies. Therefore, the present disclosure is not limited to any specific architecture. new/alternate implementations of devices are envisioned by the present disclosure.

To illustrate by way of example, computing device 12 may be a transponder (device) attached to subject 4 via a collar. A cardiac monitoring device (e.g., IMD 10) may be implanted/attached to subject 4 (e.g., a bear) and configured to transmit various physiological parameters and/or raw sensor measurements to the collar-mounted transponder via a wireless networking technology as described herein. For instance, the collar-mounted transponder may be attached to the bear's neck, and a BLUETOOTH receiver/transmitter module may be installed in both the cardiac monitoring device and the transponder to enable exchanges of various data; in this example, the distance between the bear's neck and the bear's heart is assumed to be within a pre-defined range (e.g., within 3 meters). In one example, the cardiac monitoring device may be configured with insufficient capacities of one or more resources. Instead of storing new datasets in available memory after such data is generated, the cardiac monitoring device sends each new dataset to the transponder for (temporary) storage. In one example, the transponder may have insufficient resource capacities and for at least reason, may be unable to store any new data for the bear. If the transponder fails to sense a waypoint can be sensed or otherwise, cannot transmit any of the new data or the old data, the transponder may overwrite the old data with the new data.

As the bear moves around an area (e.g., environment 28 of FIG. 1), the transponder detects network connectivity by sensing a waypoint (e.g., access point(s) 34A-34B or base station 36A) to network 16 of FIG. 1) that is located within a certain proximity. The transponder may be programmed to invoke various network functionality, in effect initiating at least one transmission of one or more of the datasets stored in its local storage. The exact distance or range at which the above transmission succeed may be established by the specific storage and networking technologies implemented by the waypoint.

Given that the capture of the bear and other wild animals to retrieve data from the cardiac monitoring device or another medical device (e.g., an implanted or wearable device) can be a difficult and expensive, the transponder essentially operates a self-powered relay for transmitting such data over long distances. To accomplish such long-distance transmissions, the transponder may be configured to transmit the retrieved data to the animal monitoring service via a cellular service or an Internet service (e.g., by way of Wi-Fi). As an option, the transponder may avail a proximately located smart phone to complete the transmission.

The transponder may be attached to the bear (as described herein) or, alternatively, located nearby (e.g., as camera 6 or ground sensor 8) and configured for remote (e.g., wireless) retrieval of monitored physiological data at known intervals. These intervals may be synced with other data collected by other sensors. As an alternative to the collar, the transponder may be enclosed in a housing (e.g., a pod) worn on a harness attached to the bear. The pod's structure to be composed of a material that allows for any radio communication and may be durable enough to be carried by the bear or even military personnel, especially given that either subject can be exposed to all types of environmental conditions. The pod may include a magnetic on-off switch for activating or deactivating the relay without disassembly. As another alternative, the transponder can be mounted in a fixed location that the animal is known to frequently visit. As an alternative to the collar or the harness, the transponder may be affixed to the bear via a backpack.

Computing device 12 may be configured with non-transitory memory units in some examples to accommodate loss or disruptions in network connectivity. In some examples, computing device 12 may be configured to receive data collected by IMD 10 and/or its own components (e.g., sensors 58). The received data is stored in the non-transitory memory units where a (e.g., permanent or non-temporary) storage/memory structure retains such data until one or more conditions trigger the automatic transmission for that data. Even if a network connection is not available, the non-transitory memory units of computing device 12 may secure the data for a substantial length of time—protecting from typical retention problems (e.g., battery exhaustion)—until the network connection becomes available. For example, computing device 12 may retain the data in non-volatile memory, which does not require an electric current to maintain its storage/memory structure.

Computing device 12 may make advantageous use of the non-volatile memory, for example, by having a smaller power source. Because the nonvolatile memory lowers the power consumption rate, computing device 12 may be configured with smaller batteries and/or manufactured to be more compact overall. As another example, computing device 12 may retain the data in flash memory, which requires current and often consumes more current to run than other memory types (e.g., volatile memory). Flash memory has advantages over the non-volatile memory, which may benefit computing device 12. In some examples, computing device 12 may realize the benefits of using flash memory by having an effective power source. Computing device 12 may be configured with a larger batter or a solar/mechanical charger; and although this configuration may cause computing device 12 to be enlarged when manufactured, the benefits of using flash memory may mitigate the larger size.

Example system 2 may employ one or more additional external devices to support or enhance (e.g., native) functionality of IMD 10 or computing device 12. Examples system 2 may establish a wireless connection, for example, to enable one-way or two-way communication between computing device 12 and another computing device of the remote computing system (e.g., computing system 20 of FIG. 1) and/or a caregiver (e.g., caregiver 26 of FIG. 1). Via the wireless connection, computing device 12 may request information in the possession of the caregiver or the remote computing system and in response to the request, the other computing device may return (in a reply) the requested information. To illustrate by way of an advantageous utility, computing device 12 may improve upon the performance of IMD 10 (e.g., with respect to health event detection), for example, by updating IMD 10 with more accurate logic (e.g., rules). For example, the caregiver may reprogram IMD 10 and/or computing device 12 using their computing device (e.g., user device 42 of FIG. 1). Event assistant 176 of computing device 12 may be configured to request new/updated criteria for rules 196 of rules engine 174 and/or rules 74 of rules engine 84. Remote reprogramming is one advantageous use of the wireless connection; the wireless connection may be put to beneficial use in a number of other ways.

To further illustrate the advantageous utility of the above wireless connection by way of another example where subject 4 benefits from such a two-way communication, event assistant 176 of computing device 12 may be configured to engage caregiver 26 in providing medical care to subject 4. Caregiver 26 may receive data for presentation on their user device 42 and based on that data, identify and/or analyze a detected health event for subject 4. For example, caregiver 26 may request a continuous stream of sensed physiological parameters to comprehend the physiology of subject 4 in response to the detected health event. As another example, event assistant 176 may provide caregiver 26 with access and/or operational control over computing device 12 and any other device in example system 2. Event assistant 176 may generate for presentation to caregiver 26 a user interface through which caregiver 26 may operate a sensor of sensors 138. Via the UI, caregiver 26 may request specific sensor data (e.g., recent measurement) to peruse on user device 42. As described herein, sensors 138 of computing device 12 may include a humidity sensor, a light sensor, a camera, a secondary electrode pair, an activity sensor (e.g., an accelerometer or a step counter), a watch mechanism for charging, and/or the like; each may generate sensed physiological data for storage in sensed data 190 and/or physiological data 194. Caregiver 26 may desire certain information associated with subject 4; to that end, via the UI, caregiver 26 may submit requests for sensed data 190 indicative of a current/previous weather for subject 4's location, a current/previous activity of subject 4 (e.g., swimming), a time of day/night recorded for an activity by subject 4, muscle activity data over a pre-determined time period (e.g., to record and enable discrimination), and/or the like. For instance, caregiver 26 may use the light sensor to track day and night in a den. Caregiver 26 may request the above sensor data to track day and night movements/activities of subject 4 (e.g., in a den). Caregiver 26 may, via UI controls and other UI components, turn on/turn off the camera as well as capture and then, immediately transmit images to user device 42. The camera may be an internal device component within computing device 12 and/or IMD 10. Alternatively, the camera may be a 360 top-view external (e.g., mounted) camera for capturing images/videos of the environment in which subject 4 is located.

As depicted in FIG. 1, example system 2 may include a number of external sensors for an environment (e.g., environment 28) of subject 4. Examples of these external sensors include mounted cameras (e.g., in camera traps), proximity sensors, telemetry stations, and/or the like. Example system 2 may include computing system 20 as a remote computing system for storing historical data for subject 4 where some of that historical data may include datasets provided by the external sensors. Computing device 12 may establish another wireless connection with computing system 20 and retrieved at least a portion of such sensor data. Each external sensor may generate sensor data and feed that data, as input data 192, to computing device 12. Event assistant 176 may present the external sensor data on the same UI provided to caregiver 26. In some examples, the external sensors may be configured to prove satisfaction of a condition described herein for triggering an upload of the external sensor data to computing system 20.

Caregiver 26 may be provided information (e.g., instructions) on initiating some action on IMD 10 and/or computing device 12. Via event assistant 176, caregiver 26 may use their user device 42 to return a rejection or a confirmation of the detected health event according to one example. For instance, caregiver 26 uses their user device 42 to return a control directive that, when received by computing device 12, causes event assistant 176 to invoke therapeutic functionality in IMD 10. If possible, event assistant 176 may communicate location data to caregiver 26 to elicit his/her assistance in caring for subject 4. If the health event is confirmed as a valid event, caregiver 26 may use their user device 42 to travel to the location of subject 4 and provide that subject 4 with medical care. Caregiver 26 may be provided instructions for administrating a medical treatment to subject 4.

Under direction of event engine 172, rules engine 174 analyzes sensed data 190, input data 192, and/or physiological data 194, to determine whether there is a sufficient likelihood that subject 4 is experiencing or is about to experience the health event detected by IMD 10. Input data 192 may include data received from IMD 10 as part of the alert transmission, additional data transmitted from IMD 10, e.g., in “real-time,” and physiological and other data related to the condition of subject 4 collected by computing device 12 and/or IoT devices 30. Input data 192 may include datasets covering a pre-determined time period of various physiological parameters. At least a portion of the input data 192 specifies one or more physiological parameter values relies upon for the health event detection. By way of storage in physiological data 194, event engine 170 may designate those parameter values for an (e.g., imminent) upload to the remote computing system.

As described herein, some examples of physiological data 194 may include one or more of: activity levels, walking/running distance, resting energy, active energy, exercise minutes, quantifications of standing, body mass, body mass index, heart rate, low, high, and/or irregular heart rate events, heart rate variability, walking heart rate, heart beat series, digitized ECG, blood oxygen saturation, blood pressure (systolic and/or diastolic), respiratory rate, maximum volume of oxygen, blood glucose, peripheral perfusion, and sleep patterns.

Input data 192 may further include human user responses to queries posed by health monitoring application 150 regarding the condition of subject 4 and other input by the human user such as caregiver 26. The queries and responses may occur responsive to the detection of the event by IMD 10, or may have occurred prior to the detection, e.g., as part long-term monitoring of the health of subject 4. Human user recorded health data may include one or more of: exercise and activity data, sleep data, symptom data, medical history data, quality of life data, nutrition data, medication taking or compliance data, allergy data, demographic data, weight, and height. Physiological data 26 may include any of the information regarding the historical condition or treatments of subject 4 described above. Physiological data 26 may relate to history of cardiac episodes, tachyarrhythmia, myocardial infarction, stroke, seizure, chronic obstructive pulmonary disease (COPD), renal dysfunction, or hypertension, history of procedures, such as ablation or cardioversion, and healthcare utilization. Physiological data 26 may also include demographic and other information of subject 4, such as age, gender, height, weight, and BMI.

Rules engine 172 may apply rules 196 to relevant datasets in data layer 156. Rules 196 may include one or more models, algorithms, decision trees, and/or thresholds. In some cases, rules 196 may be developed based on machine learning. In some examples, rules 196 and the operation of rules engine 172 may provide a more complex analysis of the data, e.g., the sensed data received from IMD 10, than is provided by rules engine 74 and rules 84. In some examples, rules 196 include one or more models developed by machine learning, and rules engine 172 applies feature vectors derived from the data to the model(s).

Rules configuration component 174 may be configured to modify rules 196 (and in some examples rules 84) based on feedback indicating whether the detections and confirmations of health events by IMD 10 and computing device 12 were accurate. The feedback may be received from the human caregiver for subject 4, or from care providers 40 via HMS 22. In some examples, rules configuration component 174 may utilize the data sets from true and false detections and confirmations for supervised machine learning to further train models included as part of rules 196.

One example rule of rules 196 may define a length of at least one time interval in a wake-up schedule for computing device 12. As an example, computing device 12 may be programmed to wake-up every five minutes. This rule may translate into one or more device settings of computing device 12 and therefore, may be modified remotely by HMS 22 or another remote computing system to either increase or decrease data transmission frequency. In addition to submitting information requests to computing device 12, caregiver 26 may submit commands directing operation of computing device 12.

As discussed above, event assistant 176 may provide a user interface for caregiver 26 to exchange information with computing device 12. Caregiver 26 may be in control of user device 42, which is connected to computing device 12, for enabling the information exchange. To the benefit of subject 4, event assistant 176 may require that caregiver 26 perform certain tasks and in some instances, caregiver 26 complies with the required tasks by invoking functionality on user device 42. User device 42 may run an application (e.g., a portal) for the user interface through which event assistant 176 presents output content and receives caregiver input. Via the user interface, event assistant 176 may enumerate instructions for caregiver 26 to follow. Event assistant 176 may request caregiver 26 return specific information that caregiver 26 knows. Event assistant 176 may query caregiver 26 regarding the condition of subject 4 in response to receiving the alert message from IMD 10. Responses from the user may be included as input data 192. Event assistant 176 may use natural language processing and context data to interpret utterances by the user. In some examples, the user utterances may confirm (or trigger) that a health event occurred or indicate that a health event detected by IMD 10 and/or other devices of system 2 did not occur. A computing device may transmit, forward, or withhold an alert message, or send a cancellation message, based on the utterances. In some examples, processing circuitry of system 2, e.g., of a computing device or HMS 22, may determine a differential diagnosis or appropriate course of care for a detected health event based on the utterances. In some examples, in addition to receiving responses to queries posed by the assistant, event assistant 176 may be configured to respond to queries posed by the user. In some examples, event assistant 176 may provide directions to and respond to queries regarding treatment of subject 4 from subject 4 or caregiver 26.

Location service 178 may determine the location of computing device 12 and, thereby, the presumed location of subject 4 (and IMD 10). Location service 178 may use global position system (GPS) data, multilateration, and/or any other known techniques for locating computing devices.

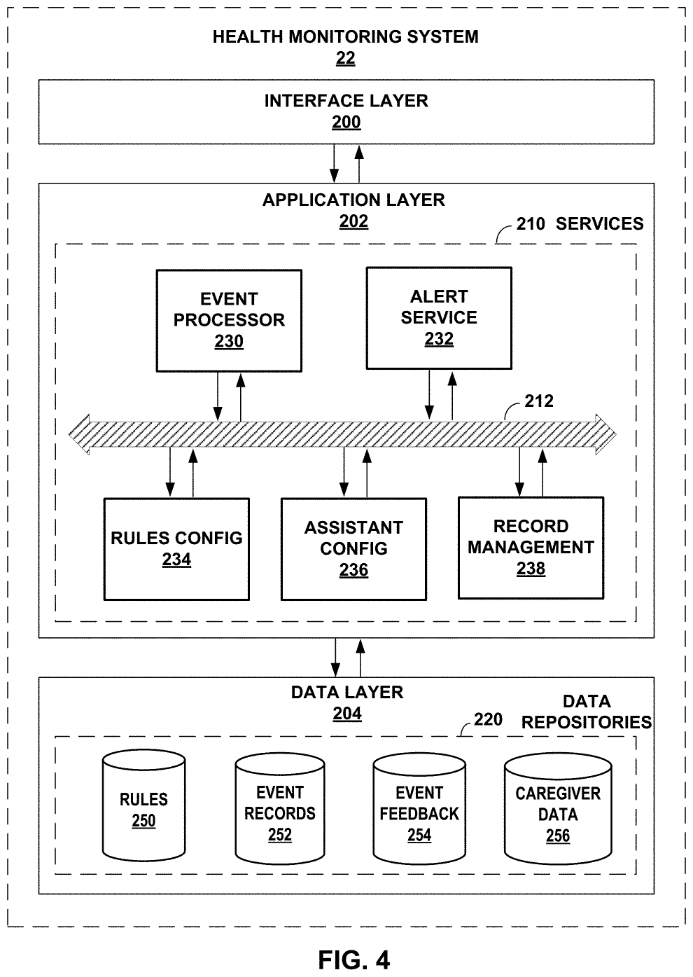

FIG. 4 is a block diagram illustrating an operating perspective of HMS 22. HMS 22 may be implemented in a computing system 20, which may include hardware components such as those of computing device 12, embodied in one or more physical devices. FIG. 4 provides an operating perspective of HMS 22 when hosted as a cloud-based platform. In the example of FIG. 4, components of HMS 22 are arranged according to multiple logical layers that implement the techniques of this disclosure. Each layer may be implemented by one or more modules comprised of hardware, software, or a combination of hardware and software.

Computing devices, such as computing devices 12, IoT devices 30, computing devices 38, and computing device 42, operate as clients that communicate with HMS 22 via interface layer 200. The computing devices typically execute client software applications, such as desktop application, mobile application, and web applications. Interface layer 200 represents a set of application programming interfaces (API) or protocol interfaces presented and supported by HMS 22 for the client software applications. Interface layer 200 may be implemented with one or more web servers.

As shown in FIG. 4, HMS 22 also includes an application layer 202 that represents a collection of services 210 for implementing the functionality ascribed to HMS herein. Application layer 202 receives information from client applications, e.g., an alert of a detected health event from a computing device 12 or IoT device 30, and further processes the information according to one or more of the services 210 to respond to the information. Application layer 202 may be implemented as one or more discrete software services 210 executing on one or more application servers, e.g., physical or virtual machines. That is, the application servers provide runtime environments for execution of services 210. In some examples, the functionality interface layer 200 as described above and the functionality of application layer 202 may be implemented at the same server. Services 210 may communicate via a logical service bus 212. Service bus 212 generally represents a logical interconnection or set of interfaces that allows different services 210 to send messages to other services, such as by a publish/subscription communication model.

Data layer 204 of HMS 22 provides persistence for information in PPEMS 6 using one or more data repositories 220. A data repository 220, generally, may be any data structure or software that stores and/or manages data. Examples of data repositories 220 include but are not limited to relational databases, multi-dimensional databases, maps, and hash tables, to name only a few examples.

As shown in FIG. 4, each of services 230-238 is implemented in a modular form within HMS 22. Although shown as separate modules for each service, in some examples the functionality of two or more services may be combined into a single module or component. Each of services 230-238 may be implemented in software, hardware, or a combination of hardware and software. Moreover, services 230-238 may be implemented as standalone devices, separate virtual machines or containers, processes, threads or software instructions generally for execution on one or more physical processors.

Event processor service 230 may be responsive to receipt of an alert transmission from computing device 12 and/or IoT device(s) 30 indicating that IMD 10 detected a health event of subject and, in some examples, that the transmitting device confirmed the detection. Event processor service 230 may initiate performance of any of the operations in response to detection of a health event ascribed herein to HMS 22, such as communicating with subject 4, caregiver 26, and care providers 40, activating drone 46 and, in some cases, analyzing data to confirm or override the detection of the health event by IMD 10.

Record management service 238 may store the subject data included in a received alert message within event records 252. Alert service 232 may package the some or all of the data from the event record, in some cases with additional information as described herein, into one more alert messages for transmission to caregiver 26 and/or care providers 40. Care giver data 256 may store data used by alert service 232 to identify to whom to send alerts based on locations of potential bystanders 26 and care givers 40 relative to a location of subject 4 and/or applicability of the care provided by care givers 40 to the health event experienced by subject 4.

In examples in which HMS 22 performs an analysis to confirm or override the detection of the health event by IMD 10, event processor service 230 may apply one or more rules 250 to the data received in the alert message, e.g., to feature vectors derived by event processor service 230 from the data. Rules 250 may include one or more models, algorithms, decision trees, and/or thresholds, which may be developed by rules configuration service 234 based on machine learning. Example machine learning techniques that may be employed to generate rules 250 can include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, Convolution Neural Networks (CNN), Long Short Term Networks (LSTM), the Apriori algorithm, K-Means Clustering, k-Nearest Neighbour (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

In some examples, in addition to rules used by HMS 22 to confirm health event detection, (or in examples in which HMS 22 does not confirm event detection) rules 250 maintained by HMS 22 may include rules 196 utilized by computing devices 12 and rules 84 used by IMD 10. In such examples, rules configuration service 234 may be configured to develop and maintain rules 196 and rules 84. Rules configuration service 234 may be configured to modify these rules based on event feedback data 254 that indicates whether the detections and confirmations of health events by IMD 10, computing device 12, and/or HMS 22 were accurate. Event feedback 254 may be received from subject 4, e.g., via computing device 12, or from care providers 40. In some examples, rules configuration service 234 may utilize event records from true and false detections (as indicated by event feedback data 254) and confirmations for supervised machine learning to further train models included as part of rules 250.

As illustrated in the example of FIG. 4, services 210 may also include an assistant configuration service 236 for configuring and interacting with event assistant 176 implemented in computing device 12 or other computing devices. For example, assistant configuration service 236 may provide event assistants updates to their natural language processing and context analyses to improve their operation over time. In some examples, assistant configuration service 236 may apply machine learning techniques to analyze sensed data and event assistant interactions stored in event records 252, as well as the ultimate disposition of the event, to modify the operation of event assistants, e.g., for particular users or devices, e.g., caregivers, bystanders, etc.

HMS 22 may be configured to monitor additional sensors for relevant sensor data to store in the historical data for subject 4. As depicted in FIG. 1, example system 2 may include a number of external sensors for an environment (e.g., environment 28) of subject 4. Examples of these external sensors include mounted cameras (e.g., in camera traps), proximity sensors, telemetry stations, and/or the like. Example system 2 may establish a wireless connection for the external sensors to transmit data including any captured sensor data for subject 4. In some examples, the external sensors may be configured to prove satisfaction of a condition described herein for triggering an upload of sensed physiological data from computing device 12 to computing system 20. In some examples, external sensors such as a camera trap, a proximity sensor, or a telemetry station may be activated by subject 4 and trigger the above-mentioned upload to computing system 20 (e.g., via a broadband or BLUETOOTH network connection).

In some examples, HMS 22 may be configured to track multiple animals with synchronous behaviors and based on their collective sensor data, trigger the above-mentioned upload to computing system. In an example where each of the multiple animals has a wearable collar-mounted transponder, HMS 22 may communicate commands directing each animal's wearable transponder to transmit an alert message to a specific care provider or caregiver or broadcast the alert message to any nearby human bystander. HMS 22 may program the transponder with rules and other logic for triggering a transmission of sensor data. HMS 22 may evaluate physiological parameters over time to determine whether a characteristic shift occurs on each of the multiple transponders and if so, triggering a transmission of sensor data. The characteristic shift is an example trigger condition and representative of unusual consistency in animal behavior indicative of an event of interest to the animals' caregiver(s).

HMS 22 may configure one or more of the multiple transponders with rules (e.g., device settings) governing when transmissions occur. This may include scheduling transmissions in advance and/or under certain criteria. In another example, HMS 22 may configure a single transponder or multiple transponders to initiate a continuous feed, for example, at arbitrary times and/or when a trigger condition is detected. In one example, a combination of factors (high heart rate, sudden acceleration, etc.) that satisfy a condition triggering the automatic transmission to computing system 20. In one example, the combination of factors may be indicative of an uncharacteristic motion (e.g., uniaxial acceleration in a vehicle versus a walking pattern) for example.

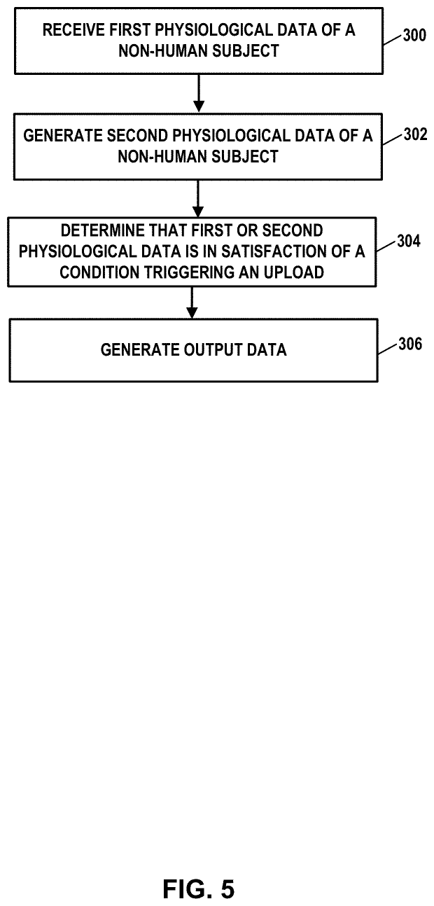

FIG. 5 is a flow diagram illustrating an example operation by a computing device of a health monitoring system that operates in accordance with one or more techniques of the present disclosure. The example operation depicted in FIG. 5 is described with respect to a computing device 12 depicted in FIGS. 1 and 3 but may be described with respect to any computing devices, e.g., computing device 12 or 38, IoT device 30, user device 42, drone 46, or health monitoring system (HMS) 22 of FIGS. 1-4 that may implement an event assistant 176.

According to the illustrated example of FIG. 5, processing circuitry of system 2 (e.g., processing circuitry 130 of one or more computing device 12) provides health event monitoring based on a subject's physiological data (e.g., sensed data 82 generated by sensing circuitry 52 of IMD 10 and/or sensed data 190 generated by sensing circuitry 136 of computing device 12). Processing circuitry of system 2 (e.g., processing circuitry 130 of one or more computing device 12) may request/receive user input (e.g., caregiver input of input data 192) for directing actions in support of the health event monitoring.

According to the illustrated example of FIG. 5, the processing circuitry detects conditions triggering an upload to a remote computing system (e.g., computing system 20 of FIG. 1) of at least a portion of the subject's physiological data including changes in the subject's health caused by a health event such as a cardiac episode. In addition to the subject's physiological data, the processing circuitry transmits location data for the subject. As described herein, the processing circuitry may communicate an alert for a detected health event to a user device (e.g., of a caregiver) and execute any action initiated by the caregiver in his/her reply to the alert (e.g., message).

To commence the non-human subject monitoring as directed by the example operation of FIG. 5, the processing circuitry receives first physiological data of a non-human subject such as a wild animal (300). The first physiological data may include monitored physiological data generated by a medical device (e.g., an implanted medical device such as IMD 10 of FIG. 1). In some examples, the medical device may be configured to monitor physiological parameters of subject 4 and generate the monitored physiological data for transmission to the computing device as at least portion of the first physiological data. In some examples, the medical device may be implanted at a position within the non-human subject where the medical device can sense the physiological parameters and provide at least a portion of the sensed physiology parameters as the first physiological data. In addition, the medical device may be configured to detect one or more conditions triggering the upload to the remote computing system.

The processing circuitry receives second physiological data of the non-human subject such as a wild animal (302). The second physiological data may include sensed physiological data generated by sensing circuitry of the same computing device as the processing circuitry. The computing device may be coupled to the non-human subject as a wearable device in which one or more (internal) sensors may be configured to sense physiological signals (e.g., parameters) as described herein.

The processing circuitry determines that the first physiological data and/or the second physiological data is in satisfaction of a condition triggering an upload of various data including at least a portion of the first physiological data and/or the second physiological data (304). In some examples and similar to the medical device, the processing circuitry may determine that the second physiological data of the subject is indicative of a health event such as a cardiac episode and then, transmit at least a portion of that data to the remote computing system for storage as a historical dataset for that subject. In some examples, the processing circuitry makes this determination based on receiving an alert message from another device, such as the medical device and/or an IOT device and then, transmit the alert message to the remote computing system for storage. For this determination, the processing circuitry may execute a confirmation procedure for evaluating the alert message (e.g., for validity) by determining whether the condition triggering the alert message was actually satisfied. The medical device may detect a health event based on an application of one or more rules (e.g., thresholds) to the first physiological data; in view of that application, the processing circuitry may be configured to apply additional criterion to the same sensed physiological parameters relied upon by the medical device and determine (e.g., confirm) that the first physiological data is indicative of the above health event.

The processing circuitry of the computing device may avail the second physiological data for the above determination including the confirmation procedure. As described herein, the sensing circuitry of the same computing device may determine the second physiological data to include values for (locally) sensed physiological parameters relied upon for triggering the upload to the remote computing system. The processing circuitry of the computing device may apply at least one second criterion to the sensed physiological parameters to decide when to perform the upload of the physiological data. In some examples, the processing circuitry may receive from the medical device an alert message indicative of a detected health event and then, based on whether the sensed physiological parameters confirm or reject the detected health event, either perform an alert cancellation or initiate some action. If the detected health event is an acute health event and/or recently occurred for the subject, the processing circuitry may transmit the alert message to the remote computing system and/or the user device of a caregiver. In response, the processing circuitry may receive a reply (message) from the remote computing system and/or the user device of a caregiver, and in accordance with that reply message, the processing circuitry may cancel the alert message, (re)program the medical device (e.g., with new/updated rules), and/or initiate another action.

For example, if the sensed physiological parameters of the computing device provide evidence that the health event is current and ongoing and has reached a certain level of severity, the processing circuitry may transmit the alert message to the remote computing system and/or the user device of a caregiver.

The above determination of the condition triggering the upload prompts the processing circuitry of the computing device to generate output data (306). As described herein, the output data may be uploaded to another computing device/system for storage as historical data. If the processing circuitry determines that a health event is occurring, has occurred, or will occur, the processing circuitry may transmit (e.g., relevant) historical or current physiological data an alert message to the remote computing system. The processing circuitry may broadcast the alert message directly to a user device of a bystander or a caregiver or, alternatively, the remote computing system may relay the alert message to the user device on behalf of the computing device. The alert message may include informational attributes describing the subject (e.g., a current location of the subject), an identity/description of the detected health event including any condition whose satisfaction triggered the alert, and any relevant physiological data. For a number of subjects, the remote computing system may make available various historical data for there are number of organizations who desire such information as well as individuals (e.g., biologists) who study and care for specific non-human subjects. If the processing circuitry decides not to suppress the alert, the processing circuitry may wirelessly broadcast the alert to network devices corresponding the remote computing system and/or the user device of a human (e.g., caregiver or bystander). In some examples, the processing circuitry may identify a new health event from the first physiological data and/or the second physiological data.

In some examples, the processing circuitry analyzes sensed physiological data to determine whether cardiac arrest is detected. As described herein, the processing circuitry may employ a rules engine (e.g., rules engine 74 and/or rules engine 172) to apply various rules (e.g., rules 84 or rules 196) to the sensed physiological data in order to determine whether a health event has occurred, is occurring, or is about to occur. In general, each rule sets forth one or more conditions (e.g., minimum or maximum thresholds, ranges, qualities, quantities, and/or other criteria for parameter values) that, if true, qualify the sensed physiological data as sufficient evidence of an imminent or an occurring health event, such as a cardiac episode.

In some examples, the medical device determines that the sensed physiological parameters of the subject are indicative of a health event such as a cardiac episode and then, communicates that indication to the computing device. In turn, the computing device may generate or trigger an alert related to the sudden cardiac arrest or the other health event. In some examples, IMD 10 and/or computing device 12 may generate an alert in the form of an auditory alarm and/or or a message communicated to a healthcare provider. In some examples, IMD 10 and/or computer device(s) 12 may trigger the alert by communicating a control directive for a third device to activate an alarm or another alert type. In some examples, IMD 10 and/or computer device(s) 12 may establish a voice or video call with a doctor, a clinician, and/or emergency medical services (EMS).

In (further) response to the determination, and based on the sensed physiological data, the remote computing system generates first audio data (e.g., output data 198 of FIG. 3) and then, transmits data causing the user device to output a first plurality of utterances representing a query related to the health event. In general, the processing circuitry may configure the first audio data to engage in a conversation with the caregiver (e.g., to explore what the caregiver could do) and possibly, provides some closed-loop emotional/humanistic engagement experience. To that end, the first plurality of utterances may be in the form of a question for the caregiver or another user and include one or more words designed to elicit intelligence, including information that cannot be ascertained from the sensed physiological data (e.g., without difficulty). Any gained intelligence may be incorporated in an application of one or more rules, possibly resulting in a different determination or therapy.

System 2 may employ a number of technologies (e.g., the caregiver's mobile phone, a smart speaker device, and/or IMD 10 itself) to engage with the caregiver, for example, to assisting in assess the subject's condition and providing at least some medical care. System 2 (e.g., via health monitoring application 150) may generate a vocal representation of the query from the first plurality of utterances. In combination with the vocal representation, the output device may also display, on an electronic display, a textual representation of the first set of utterances, allowing the caregiver to fully comprehend the query. In some examples, the output device may utilize one or more modes to present the alert of the health event, for example, contemporaneous with the first audio data.

The user device may output the first audio data to present information related to the health event determined from the physiological data, prompt the caregiver for initial input, return that initial input to the remote computing system. As part of the caregiver's initial input, the caregiver may proceed to describe current or past symptoms. Based on that initial input, the health management system of the remote computing system may initiate an action for the health event including determining whether the health event is accurate.

The health management system of the remote computing system receives from the user device second audio data that represents a second plurality of utterances of at least one of the caregiver or another user subsequent to the query. The second audio data may include the caregiver's response to the query, including answer(s) to any question(s) regarding the subject's health. After receiving the response from the caregiver or the other user, the processing circuitry generates output data based on the sensed physiological data and application of natural language processing to the second plurality of utterances. As described herein, the event assistant may employ a number of speech-recognition technologies to perform the natural language processing of the second plurality of utterances.

The first plurality of utterances and the second plurality of utterances may form at least a portion of a conversation between the patient or other user and event assistant 176. The sensed physiological data, as described herein, may be used to evaluate one or more rules for determining whether the subject is experiencing a health event. The conversation (particularly, the caregiver's response) may provide information to evaluate additional or new rules, ensuring a higher level of accuracy in the determination regarding the health event. In some instances, as a result of the second plurality of utterances, the processing circuitry may modify a previous determination of the health event. The processing circuitry of the computing device or the processing circuitry of the remote computing system, in turn, may generate output data indicative of a modified determination based on the application of natural language processing to the second plurality of utterances. The modified determination may indicate a false rejection, a false detection, a true rejection, or a true rejection of the health event. In some examples, based on the application of natural language processing to the second plurality of utterances, the processing circuitry computes or modifies a likelihood of the health event based on the sensed physiological data. The processing circuitry of may update a likelihood probability (e.g., a prior probability or a conditional probability) of the health event based on the modified determination.

To further illustrate, via the output device and/or another device, the processing circuitry may trigger an ongoing alert (e.g., alarm) indicating that an emergency has occurred and one or more emergency responders have been summoned. In other examples, the processing circuitry of may cancel the ongoing alert of the health event. In addition to the cancellation of the alarm, the processing circuitry may generate and then, communicate to, one or more healthcare providers, messages conveying that the subject is not experiencing the health event. An example message may indicate that the alarm was a false alarm, or that the sudden cardiac arrest determination is a true detection but that the subject has recovered or is stable. An example message may prompt a user or device to alter the subject's therapy (e.g., to start/stop CPR). Another example message may prompt a user or device to remove a specific treatment. Another example message may be communicated to the EMS in order to redirect an ambulance to a specialized care center or another caregiver. Another example message may be communicated to a caregiver to trigger a specific treatment (for example, instruct the caregiver/bystander 42 to utilize an AED, find the location of a nearby drone-delivered AED, take medication or another treatment, and/or the like) and then, add that specific treatment to the subject's therapy.

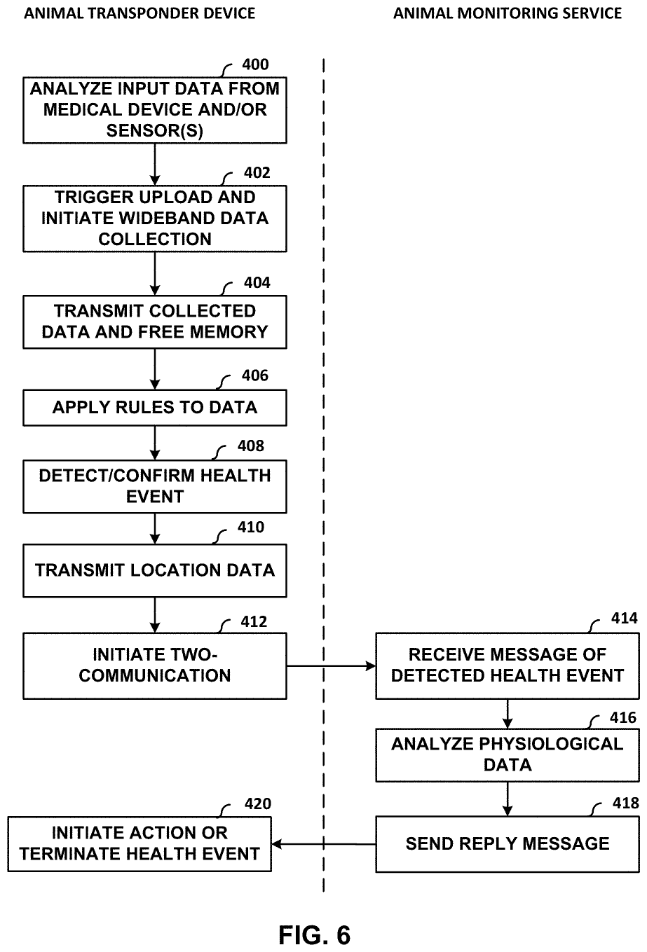

FIG. 6 is a flow diagram illustrating an example interactive operation between an animal transponder device and an animal monitoring service that each operate in accordance with one or more techniques of the present disclosure. Although described with respect to computing device 12 as the animal transponder device and HMS 22 as the animal monitoring service, the example of FIG. 6 may be performed by any computing device including computing device 38 or 42, IoT device(s) 30, or drone 46. The animal transponder device and computing devices for the animal monitoring service form at least a portion of system 2 and the example operation depicted in FIG. 6 may be described with respect to system 2 of FIGS. 1-4.

According to the illustrated example of FIG. 6, system 2 implements technology enabling interaction (e.g., by way of an exchange of messages) between the animal transponder device and the animal monitoring service. In this manner, the animal transponder device may leverage resources (e.g., hardware capacity and/or software capability) of the animal monitoring service to enhance native functionality.

In the example of FIG. 6, processing circuitry of the animal transponder device (e.g., processing circuitry 130 of computing device 12) analyzes input data from a medical device and/or one or more sensors (400). It should be noted that the animal transponder device may be communicatively coupled to a number of different sensors, and one or more device components may be configured to make advantageous use of sensed data. Among the available sensors for sensing relevant information, one or more sensors may be internal, attached, and/or connected by wire to sensing circuitry (e.g., sensing circuitry 136 of computing device 12 of FIG. 3) of the animal transponder device. Another sensor may be wirelessly connected to the animal transponder device.

The processing circuitry of the animal transponder device triggers an upload of stored datasets and initiates wideband data collection at the medical device and any other device having relevant information (402). The processing circuitry of the animal transponder device may continue the upload by transmitting any collected data and then, to store more (e.g., new) data, free memory space previously allocated to the uploaded data (404). A number of conditions may be configured into logic executed by the processing circuitry of the animal transponder device.

The input data stored in the memory of the animal transponder device includes monitored physiological data from the medical device and sensed physiological data from the one or more sensors, and the processing circuitry may run a rules engine configured to apply rules to the stored input data (406).

The processing circuitry of the animal transponder device may detect a health event for the animal and as an option, execute a confirmation procedure to determine whether to confirm or reject the health event (408). As another option, the animal monitoring service may be configured to execute an independent confirmation procedure to determine whether to confirm or reject the health event. In some examples, the animal transponder device receives, via communication circuitry, an alert message from the medical device and the processing circuitry may process that alert message to identify a health event detected by the medical device. The processing circuitry may confirm the health event with sensed physiological data. In other examples, the processing circuitry may access the stored data and analyze that data using rules for detecting health events. The processing circuitry may confirm the health event with additional (e.g., current) physiological data.

The processing circuitry of the animal transponder device may transmit location data for the animal to any (authorized) receiving device (410). As described herein, the animal transponder device may instrument a GPS module to receive GPS coordinates for the animal having the health event and then, communicate that location data to a computing device of the animal monitoring service, a computing device of a healthcare provider, and/or a user device of a specific caregiver for that animal. The animal transponder device may broadcast the alert message for the detected health event to any bystander or caregiver user device in the same environment as the animal.

The processing circuitry of the animal transponder device may decide to initiate a two-way communication with the receiving device (412), which FIG. 6 illustrates as a computing device of the animal monitoring service. In turn, processing circuitry of the computing device of the animal monitoring service receives a message indicative of the detected health event. In addition to identifying the detected health event, the message may include relevant information including the physiological parameter values using in (e.g., a rule for) detecting the health event.

The computing device of the animal monitoring service stores historical data for the animal being monitored and may request/retrieve additional sensor data from one or more sensors (e.g., in environment 28). As described herein, the historical data includes sequential datasets of physiological parameter values that have been uploaded over time. Combining the multiple courses of physiological parameter values, the processing circuitry of the computing device of the animal monitoring service may analyze that physiological data for a number of target data points (416). In some examples, the computing device of the animal monitoring service may run a validation process for the detected health event. In this manner, the computing device may apply the latest techniques for that particular animal. In some examples, the computing device of the animal monitoring service may determine that immediate medical care benefits the animal and inform a caregiver of the detected health event. In some examples, the animal monitoring service may request an emergency medical service to provide the animal with imminent treatment(s).

As illustrated in FIG. 6, the animal monitoring service may be directed to initiate some action on the animal transponder device and/or the medical device coupled to the animal transponder device. The above computing device or another computing device of the animal monitoring service may send a reply (message) in return to the message of the detected health event (418). As a response to receiving the reply message, the processing circuitry of the animal transponder device may initiate the action identified in the reply message or terminate the health event (420). In this manner, the previous alert for the health event is cancelled or suppressed.

To illustrate an example action to initiate, the processing circuitry of the computing device of the animal monitoring service may determine a method and data for updating the logic employed in the animal transponder device and/or the associated medical device. The method enumerates functions for the processing circuitry of the animal transponder device to invoke functions in order to reprogram rules of the rule engine or another logical element (e.g., program code) configured to detect the health event.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

Various examples have been described. These and other examples are within the scope of the following claims.

Example 1: A computing device communicatively coupled to a medical device of a non-human subject includes processing circuitry; and a memory includes receive monitored physiological data from the medical device configured to monitor the physiological data of the non-human subject; determine whether the medical device indicates that the monitored physiological data is in satisfaction of at least one condition triggering an upload of at least a portion of the monitored physiological data to a remote computing system configured to store historical data for the non-human subject; and in response to the determination, generate, for communication to the remote computing system, output data comprising the at least a portion of the monitored physiological data.

Example 2: The computing device of example 1 further comprising within a housing affixed to the non-human subject via at least one of a harness or a collar, wherein the processing circuitry and the memory are contained within the housing.

Example 3: The computing device of any of examples 1 and 2, wherein the instructions further cause the processing circuitry to: in response to a determination that the monitored physiological data of the non-human subject is indicative of a health event, generate, for communication to the remote computing system, output data comprising an indication of the health event, wherein the remote computing system is configured to store the output data as historical data for the non-human subject.

Example 4: The computing device of any of examples 1 through 3, wherein the instructions further cause the processing circuitry to: determine that, for a heart of the non-human subject, the medical device indicates an occurrence of an episode of an arrhythmia or another cardiac dysfunction, an abnormal behavior movement, or another medical emergency.

Example 5: The computing device of any of examples 1 through 4, wherein the instructions further cause the processing circuitry to: determine at least one of a heart rate measurement exceeds a first threshold, an impedance measurement exceeds a second threshold, a temperature measurement exceeds a third threshold, a change in temperature per unit of time exceeds a fourth threshold, accelerometer data satisfies a fifth threshold, a respiration rate exceeds a sixth threshold, or an oxygen saturation satisfies a seventh threshold.

Example 6: The computing device of any of examples 1 through 5, wherein the memory further comprises instructions that, when executed by the processing circuitry, cause the processing circuitry to: communicate, to a user device, a message comprising the output data; and receive, from the user device, a reply comprising a control directive to initiate an action.

Example 7: The computing device of any of examples 1 through 6, wherein the memory further comprises instructions that, when executed by the processing circuitry, cause the processing circuitry to: communicate, to a user device, an alert of a health event for the non-human subject; and receive, from the user device, a reply comprising a confirmation or a termination of the health event.

Example 8: The computing device of example 7, wherein the reply further comprises one or more parameter values for reprogramming the medical device.

Example 9: The computing device of any of examples 1 through 8 further includes sensing circuitry coupled to one or more sensors and configured to generate sensed physiological data; wherein the memory further comprises instructions that, when executed by the processing circuitry, cause the processing circuitry to: determine that the sensed physiological data is in satisfaction of the at least one condition triggering an upload of at least a portion of the sensed physiological data; and in response to the determination, generate, for communication to the remote computing system, second output data comprising the at least a portion of the sensed physiological data.

Example 10: The computing device of example 9, wherein the instructions further cause the processing circuitry to: in response to a determination that at least one of the sensed physiological data of the non-human subject is indicative of a health event, generate the second output data comprising an indication of the health event.

Example 11: The computing device of any of examples 9 and 10, wherein the instructions further cause the processing circuitry to: determine, from the sensed physiological data, at least one of movement data satisfies at least one first criterion, at least one of altitude data or location data satisfies at least one second criterion, audio data of the non-human subject satisfies at least one third criterion, or image data of the non-human subject satisfies at least one fourth criterion.

Example 12: The computing device of any of examples 9 through 11, wherein the instructions further cause the processing circuitry to: based on the sensed physiological data or the communication circuitry, detect a disconnection with the medical device.

Example 13: The computing device of any of examples 9 through 12, wherein the instructions further cause the processing circuitry to: in response to detecting network access from signal data of a network device that is communicatively coupled to the remote computing system, generate, for communication to the network device, the output data comprising at least one of the at least a portion of the monitored physiological data or the at least one portion of the sensed physiological data.

Example 14: The computing device of any of examples 9 through 13, wherein the instructions further cause the processing circuitry to: in response to an insufficient resource capacity, generate, for communication to a network device, the output data comprising at least one of the at least a portion of the monitored physiological data or the at least one portion of the sensed physiological data.

Example 15: The computing device of any of examples 1 through 14, wherein to receive the monitored physiological data, the processing circuitry is configured to: retrieve at least a portion of the monitored physiological data in accordance with a schedule established by the remote monitoring system or in accordance with an information request received from the remote monitoring system.

Example 16: The computing device of any of examples 1 through 15, further includes based on a determination that a network device is available for transmitting data to the remote computing system, perform, via the communication circuitry, at least one of retrieve, from the medical device a portion of the monitored physiological data or communicate, to the remote computing system, the output data or second output data comprising the at least a portion of the monitored physiological data.

Example 17: The computing device of example 16 wherein to determine availability of the communication network, the processing circuitry is configured to at least one of: broadcast a polling message for detecting a network device of the communication network within an area; listen for a second polling message or an acknowledgment of the first polling message from a network device of the communication network within the area; or determine whether location data indicates a network device of the communication network is within an effective distance for receiving a data transmission based on receiving at least one of the second polling message or the acknowledgment of the first polling message comprising the location data.

Example 18: The computing device of any of examples 16 and 17 wherein to determine availability of the medical device for data transmission, the processing circuitry is configured to: in response to determination that the communication network is available, transition between a first power mode and a second power mode, wherein the second power mode consumes less power than the first power mode or the second power mode consumes more power than the first power mode.

Example 19: A medical device for a non-human subject includes one or more sensors configured to sense one or more physiological signals of the non-human subject; sensing circuitry configured to generate physiological data based on the one or more sensed physiological signals; communication circuitry configured to be communicatively coupled to a computing device, wherein the computing device is coupled to the non-human subject or a user; processing circuitry; and a memory includes determine that the physiological data is in satisfaction of at least one condition triggering an upload of the physiological data, wherein the at least one condition comprises a threshold defining a minimum acceleration; in response to the determination that accelerometer data satisfies at least one threshold, generate, for communication to the computing device, output data comprising the physiological data.

Example 20: The medical device of example 19, wherein to determine that the physiological data is in satisfaction of the at least one condition triggering the upload, the instructions cause the processing circuitry to: in response to a determination that the sensed physiological data is indicative of a cardiac episode for the non-human subject, generate, for communication to the computing device, output data comprising the cardiac episode and at least a portion of the sensed physiological data.

Example 21: The medical device of any of examples 19 and 20, wherein the memory further comprises instructions that, when executed by the processing circuitry, cause the processing circuitry to: in response to an insufficient resource capacity, generate, for communication to a network device, the output data comprising the monitored physiological data.

Example 22: The medical device of any of examples 19 through 21 further comprising an implantable medical device or a wearable medical device.

Example 23: A method performed by processing circuitry of a computing device communicatively coupled to a medical device for a non-human subject includes receiving physiological data from the medical device, the medical device configured to monitor the physiological data of the non-human subject; determining whether the monitored physiological data is in satisfaction of at least one condition triggering an upload of at least a portion of the monitored physiological data to a remote computing system configured to store historical data for the non-human subject; and in response to determining that the monitored physiological data is in satisfaction of at least one condition, generating, for communication to the remote computing system via communication circuitry, output data comprising the at least a portion of the monitored physiological data.

Example 24: The method of example 23, wherein generating the output data further comprises: generating, by sensing circuitry coupled to one or more sensors, sensed physiological data of the non-human subject; in response to determining that the sensed physiological data is in satisfaction of the at least one condition triggering an upload of at least a portion of the sensed physiological data to the remote computing system, generate, for communication to the remote computing system via the communication circuitry, second output data comprising the at least a portion of the sensed physiological data.

What is claimed is:

1. A computing device communicatively coupled to a medical device of a non-human subject comprising:
processing circuitry; and
a memory comprising instructions that, when executed by the processing circuitry, cause the processing circuitry to:
receive monitored physiological data from the medical device configured to monitor the physiological data of the non-human subject;
determine whether the medical device indicates that the monitored physiological data is in satisfaction of at least one condition triggering an upload of at least a portion of the monitored physiological data to a remote computing system configured to store historical data for the non-human subject; and
in response to the determination, generate, for communication to the remote computing system, output data comprising the at least a portion of the monitored physiological data.

2. The computing device of claim 1 further comprising within a housing affixed to the non-human subject via at least one of a harness or a collar, wherein the processing circuitry and the memory are contained within the housing.

3. The computing device of claim 1, wherein the instructions further cause the processing circuitry to:
in response to a determination that the monitored physiological data of the non-human subject is indicative of a health event, generate, for communication to the remote computing system, output data comprising an indication of the health event, wherein the remote computing system is configured to store the output data as historical data for the non-human subject.

4. The computing device of claim 1, wherein the instructions further cause the processing circuitry to:
determine that, for a heart of the non-human subject, the medical device indicates an occurrence of an episode of an arrhythmia or another cardiac dysfunction, an abnormal behavior movement, or another medical emergency.

5. The computing device of claim 1, wherein the instructions further cause the processing circuitry to:
determine at least one of a heart rate measurement exceeds a first threshold, an impedance measurement exceeds a second threshold, a temperature measurement exceeds a third threshold, a change in temperature per unit of time exceeds a fourth threshold, accelerometer data satisfies a fifth threshold, a respiration rate exceeds a sixth threshold, or an oxygen saturation satisfies a seventh threshold.

6. The computing device of claim 1, wherein the memory further comprises instructions that, when executed by the processing circuitry, cause the processing circuitry to:
communicate, to a user device, a message comprising the output data; and
receive, from the user device, a reply comprising a control directive to initiate an action.

7. The computing device of claim 1, wherein the memory further comprises instructions that, when executed by the processing circuitry, cause the processing circuitry to:
communicate, to a user device, an alert of a health event for the non-human subject; and
receive, from the user device, a reply comprising a confirmation or a termination of the health event.

8. The computing device of claim 7, wherein the reply further comprises one or more parameter values for reprogramming the medical device.

9. The computing device of claim 1 further comprising:
sensing circuitry coupled to one or more sensors and configured to generate sensed physiological data;
wherein the memory further comprises instructions that, when executed by the processing circuitry, cause the processing circuitry to:
determine that the sensed physiological data is in satisfaction of the at least one condition triggering an upload of at least a portion of the sensed physiological data; and
in response to the determination, generate, for communication to the remote computing system, second output data comprising the at least a portion of the sensed physiological data.

10. The computing device of claim 9, wherein the instructions further cause the processing circuitry to:
in response to a determination that at least one of the sensed physiological data of the non-human subject is indicative of a health event, generate the second output data comprising an indication of the health event.

11. The computing device of claim 9, wherein the instructions further cause the processing circuitry to:
determine, from the sensed physiological data, at least one of movement data satisfies at least one first criterion, at least one of altitude data or location data satisfies at least one second criterion, audio data of the non-human subject satisfies at least one third criterion, or image data of the non-human subject satisfies at least one fourth criterion.

12. The computing device of claim 9, wherein the instructions further cause the processing circuitry to:
based on the sensed physiological data or the communication circuitry, detect a disconnection with the medical device.

13. The computing device of claim 9, wherein the instructions further cause the processing circuitry to:
in response to detecting network access from signal data of a network device that is communicatively coupled to the remote computing system, generate, for communication to the network device, the output data comprising at least one of the at least a portion of the monitored physiological data or the at least one portion of the sensed physiological data.

14. The computing device of claim 9, wherein the instructions further cause the processing circuitry to:
in response to an insufficient resource capacity, generate, for communication to a network device, the output data comprising at least one of the at least a portion of the monitored physiological data or the at least one portion of the sensed physiological data.

15. The computing device of claim 1, wherein to receive the monitored physiological data, the processing circuitry is configured to:
retrieve at least a portion of the monitored physiological data in accordance with a schedule established by the remote monitoring system or in accordance with an information request received from the remote monitoring system.

16. The computing device of claim 1, further comprising
communication circuitry configured to communicatively couple the computing device with the medical device and the remote computing system;
wherein the instructions further cause the processing circuitry to:
based on a determination that a network device is available for transmitting data to the remote computing system, perform, via the communication circuitry, at least one of retrieve, from the medical device a portion of the monitored physiological data or communicate, to the remote computing system, the output data or second output data comprising the at least a portion of the monitored physiological data.

17. The computing device of claim 16 wherein to determine availability of the communication network, the processing circuitry is configured to at least one of:
broadcast a polling message for detecting a network device of the communication network within an area;
listen for a second polling message or an acknowledgment of the first polling message from a network device of the communication network within the area; or
determine whether location data indicates a network device of the communication network is within an effective distance for receiving a data transmission based on receiving at least one of the second polling message or the acknowledgment of the first polling message comprising the location data.

18. The computing device of claim 16 wherein to determine availability of the medical device for data transmission, the processing circuitry is configured to:
in response to determination that the communication network is available, transition between a first power mode and a second power mode, wherein the second power mode consumes less power than the first power mode or the second power mode consumes more power than the first power mode.

19. A medical device for a non-human subject comprising:
one or more sensors configured to sense one or more physiological signals of the non-human subject;
sensing circuitry configured to generate physiological data based on the one or more sensed physiological signals;
communication circuitry configured to be communicatively coupled to a computing device, wherein the computing device is coupled to the non-human subject or a user;
processing circuitry; and
a memory comprising instructions that, when executed by the processing circuitry, cause the processing circuitry to:
determine that the physiological data is in satisfaction of at least one condition triggering an upload of the physiological data, wherein the at least one condition comprises a threshold defining a minimum acceleration;
in response to the determination that accelerometer data satisfies at least one threshold, generate, for communication to the computing device, output data comprising the physiological data.

20. The medical device of claim 19, wherein to determine that the physiological data is in satisfaction of the at least one condition triggering the upload, the instructions cause the processing circuitry to:
in response to a determination that the sensed physiological data is indicative of a cardiac episode for the non-human subject, generate, for communication to the computing device, output data comprising the cardiac episode and at least a portion of the sensed physiological data.

21. The medical device of claim 19, wherein the memory further comprises instructions that, when executed by the processing circuitry, cause the processing circuitry to:
in response to an insufficient resource capacity, generate, for communication to a network device, the output data comprising the monitored physiological data.

22. The medical device of claim 19 further comprising an implantable medical device or a wearable medical device.

23. A method performed by processing circuitry of a computing device communicatively coupled to a medical device for a non-human subject, the method comprising:
receiving physiological data from the medical device, the medical device configured to monitor the physiological data of the non-human subject;
determining whether the monitored physiological data is in satisfaction of at least one condition triggering an upload of at least a portion of the monitored physiological data to a remote computing system configured to store historical data for the non-human subject; and
in response to determining that the monitored physiological data is in satisfaction of at least one condition, generating, for communication to the remote computing system via communication circuitry, output data comprising the at least a portion of the monitored physiological data.

24. The method of claim 23, wherein generating the output data further comprises:
generating, by sensing circuitry coupled to one or more sensors, sensed physiological data of the non-human subject;
in response to determining that the sensed physiological data is in satisfaction of the at least one condition triggering an upload of at least a portion of the sensed physiological data to the remote computing system, generate, for communication to the remote computing system via the communication circuitry, second output data comprising the at least a portion of the sensed physiological data.
